SEQUENCE LISTING (36 sequences)

SEQ ID NO: 1 - DNA, 3706 bp, Burkholderia cepacia
  source 1..3706: mol_type = genomic DNA
  CDS 258..761: protein_id = 2; translation = MHNDNTPHSRRHGDAAASGITRRQWLQGALALTAAGLTGSLTLR ALADNPGTAPLDTFMTLSESLTGKKGLSRVIGERLLQALQKGSFKTADSLPQLAGALA SGSLTPEQESLALTILEAWYLGIVDNVVITYEEALMFGVVSDTLVIRSYCPNKPGFWA DKPIERQA
  CDS 764..2380: protein_id = 3; translation = MADTDTQKADVVVVGSGVAGAIVAHQLAMAGKAVILLEAGPRMP RWEIVERFRNQPDKMDFMAPYPSSPWAPHPEYGPPNDYLILKGEHKFNSQYIRAVGGT TWHWAASAWRFIPNDFKMKSVYGVGRDWPIQYDDLEPYYQRAEEELGVWGPGPEEDLY SPRKQPYPMPPLPLSFNEQTIKTALNNYDPKFHVVTEPVARNSRPYDGRPTCCGNNNC MPICPIGAMYNGIVHVEKAERAGAKLIENAVVYKLETGPDKRIVAALYKDKTGAEHRV EGKYFVLAANGIETPKILLMSANRDFPNGVANSSDMVGRNLMDHPGTGVSFYASEKLW PGRGPQEMTSLIGFRDGPFRATEAAKKIHLSNLSRIDQETQKIFKAGKLMKPDELDAQ IRDRSARYVQFDCFHEILPQPENRIVPSKTATDAIGIPRPEITYAIDDYVKRGAAHTR EVYATAAKVLGGTDVVFNDEFAPNNHITGSTIMGADARDSVVDKDCRTFDHPNLFISS SATMPTVGTVNVTLTIAALALRMSDTLKKEV
  CDS 2386..3660: protein_id = 4; translation = VRKSTLTFLIAGCLALPGFARAADAADPALVKRGEYLATAGDCM ACHTVKGGKPYAGGLGMPVPMLGKIYTSNITPDPDTGIGKWTFEDFERAVRHGVSKNG DNLYPAMPYVSYAKITDDDVRALYAYFMHGVEPVKQAPPKNEIPALLSMRWPLKIWNW LFLKDGPYQPKPSQSAEWNRGAYLVQGLAHCSTCHTPRGIAMQEKSLDETGGSFLAGS VLAGWDGYNITSDPNAGIGSWTQQQLVQYLRTGSVPGVAQAAGPMAEAVEHSFSKMTE ADIGAIATYVRTVPAVADSNAKQPRSSWGKPAEDGLKLRGVALASSGIDPARLYLGNC ATCHQMQGKGTPDGYYPSLFHNSTVGASNPSNLVQVILNGVQRKIGSEDIGMPAFRYD LNDAQIAALTNYVTAQFGNPAAKVTEQDVAKLR
aagctttctgtttgattgcacgcgattctaaccgagcgtctgtgaggcggaacgcgacatgcttcgtgtcgcacacgtgtcgcgccgacgacacaaaaatgcagcgaaatggctgatcgttacgaatggctgacacattgaatggactataaaaccattgtccgttccggaatgtgcgcgtacatttcaggtccgcgccgatttttgagaaatatcaagcgtggttttcccgaatccggtgttcgagagaaggaaacatgcacaacgacaacactccccactcgcgtcgccacggcgacgcagccgcatcaggcatcacgcggcgtcaatggttgcaaggcgcgctggcgctgaccgcagcgggcctcacgggttcgctgacattgcgggcgcttgcagacaaccccggcactgcgccgctcgatacgttcatgacgctttccgaatcgctgaccggcaagaaagggctcagccgcgtgatcggcgagcgcctgctgcaggcgctgcagaagggctcgttcaagacggccgacagcctgccgcagctcgccggcgcgctcgcgtccggttcgctgacgcctgaacaggaatcgctcgcactgacgatcctcgaggcctggtatctcggcatcgtcgacaacgtcgtgattacgtacgaggaagcattaatgttcggcgtcgtgtccgatacgctcgtgatccgttcgtattgccccaacaaacccggcttctgggccgacaaaccgatcgagaggcaagcctgatggccgataccgatacgcaaaaggccgacgtcgtcgtcgttggatcgggtgtcgcgggcgcgatcgtcgcgcatcagctcgcgatggcgggcaaggcggtgatcctgctcgaagcgggcccgcgcatgccgcgctgggaaatcgtcgagcgcttccgcaatcagcccgacaagatggacttcatggcgccgtacccgtcgagcccctgggcgccgcatcccgagtacggcccgccgaacgactacctgatcctgaagggcgagcacaagttcaactcgcagtacatccgcgcggtgggcggcacgacgtggcactgggccgcgtcggcgtggcgcttcattccgaacgacttcaagatgaagagcgtgtacggcgtcggccgcgactggccgatccagtacgacgatctcgagccgtactatcagcgcgcggaggaagagctcggcgtgtggggcccgggccccgaggaagatctgtactcgccgcgcaagcagccgtatccgatgccgccgctgccgttgtcgttcaacgagcagaccatcaagacggcgctgaacaactacgatccgaagttccatgtcgtgaccgagccggtcgcgcgcaacagccgcccgtacgacggccgcccgacttgttgcggcaacaacaactgcatgccgatctgcccgatcggcgcgatgtacaacggcatcgtgcacgtcgagaaggccgaacgcgccggcgcgaagctgatcgagaacgcggtcgtctacaagctcgagacgggcccggacaagcgcatcgtcgcggcgctctacaaggacaagacgggcgccgagcatcgcgtcgaaggcaagtatttcgtgctcgccgcgaacggcatcgagacgccgaagatcctgctgatgtccgcgaaccgcgatttcccgaacggtgtcgcgaacagctcggacatggtcggccgcaacctgatggaccatccgggcaccggcgtgtcgttctatgcgagcgagaagctgtggccgggccgcggcccgcaggagatgacgtcgctgatcggtttccgcgacggtccgttccgcgcgaccgaagcggcgaagaagatccacctgtcgaacctgtcgcgcatcgaccaggagacgcagaagatcttcaaggccggcaagctgatgaagcccgacgagctcgacgcgcagatccgcgaccgttccgcacgctacgtgcagttcgactgcttccacgaaatcctgccgcaacccgagaaccgcatcgtgccgagcaagacggcgaccgatgcgatcggcattccgcgccccgagatcacgtatgcgatcgacgactacgtgaagcgcggcgccgcgcatacgcgcgaggtctacgcgaccgccgcgaaggtgctcggcggcacggacgtcgtgttcaacgacgaattcgcgccgaacaatcacatcacgggctcgacgatcatgggcgccgatgcgcgcgactccgtcgtcgacaaggactgccgcacgttcgaccatccgaacctgttcatttcgagcagcgcgacgatgccgaccgtcggtaccgtaaacgtgacgctgacgatcgccgcgctcgcgctgcggatgtcggacacgctgaagaaggaagtctgaccgtgcggaaatctactctcactttcctcatcgccggctgcctcgcgttgccgggcttcgcgcgcgcggccgatgcggccgatccggcgctggtcaagcgcggcgaatacctcgcgaccgccggcgactgcatggcctgccacaccgtgaagggcggcaagccgtacgcgggcggccttggcatgccggtaccgatgctcggcaagatctacacgagcaacatcacgcccgatcccgatacgggcatcggcaaatggacgttcgaggacttcgagcgcgcggtgcggcacggcgtgtcgaagaacggcgacaacctgtatccggcgatgccgtacgtgtcgtacgcgaagatcacggacgacgacgtacgcgcgctgtacgcctacttcatgcacggcgtcgagccggtcaagcaggcgccgccgaagaacgagattcccgcgctgctcagcatgcgctggccgctgaagatctggaactggctgttcctgaaggacggcccgtaccagccgaagccgtcgcagagcgccgaatggaatcgcggcgcgtatctggtgcagggtctcgcgcactgcagcacgtgccacacgccgcgcggcatcgcgatgcaggagaagtcgctcgacgaaaccggcggcagcttcctcgcggggtcggtgctcgccggctgggacggctacaacatcacgtcggacccgaatgcggggatcggcagctggacgcagcagcagctcgtgcagtatttgcgcaccggcagcgtgccgggcgtcgcgcaggcggccgggccgatggccgaggcggtcgagcacagcttctcgaagatgaccgaagcggacatcggtgcgatcgccacgtacgtccgcacggtgccggccgttgccgacagcaacgcgaagcagccgcggtcgtcgtggggcaagccggccgaggacgggctgaagctgcgcggtgtcgcgctcgcgtcgtcgggcatcgatccggcgcggctgtatctcggcaactgcgcgacgtgccaccagatgcagggcaagggcacgccggacggctattacccgtcgctgttccacaactccaccgtcggcgcgtcgaatccgtcgaacctcgtgcaggtgatcctgaacggcgtgcagcgcaagatcggcagcgaggatatcgggatgcccgctttccgctacgatctgaacgacgcgcagatcgccgcgctgacgaactacgtgaccgcgcagttcggcaatccggcggcgaaggtgacggagcaggacgtcgcgaagctgcgctgacatagtcgggcgcgccgacacggcgcaaccgataggacaggag

SEQ ID NO: 2 - protein, 168 aa, Burkholderia cepacia
MHNDNTPHSRRHGDAAASGITRRQWLQGALALTAAGLTGSLTLRALADNPGTAPLDTFMTLSESLTGKKGLSRVIGERLLQALQKGSFKTADSLPQLAGALASGSLTPEQESLALTILEAWYLGIVDNVVITYEEALMFGVVSDTLVIRSYCPNKPGFWADKPIERQA

SEQ ID NO: 3 - protein, 539 aa, Burkholderia cepacia
MADTDTQKADVVVVGSGVAGAIVAHQLAMAGKAVILLEAGPRMPRWEIVERFRNQPDKMDFMAPYPSSPWAPHPEYGPPNDYLILKGEHKFNSQYIRAVGGTTWHWAASAWRFIPNDFKMKSVYGVGRDWPIQYDDLEPYYQRAEEELGVWGPGPEEDLYSPRKQPYPMPPLPLSFNEQTIKTALNNYDPKFHVVTEPVARNSRPYDGRPTCCGNNNCMPICPIGAMYNGIVHVEKAERAGAKLIENAVVYKLETGPDKRIVAALYKDKTGAEHRVEGKYFVLAANGIETPKILLMSANRDFPNGVANSSDMVGRNLMDHPGTGVSFYASEKLWPGRGPQEMTSLIGFRDGPFRATEAAKKIHLSNLSRIDQETQKIFKAGKLMKPDELDAQIRDRSARYVQFDCFHEILPQPENRIVPSKTATDAIGIPRPEITYAIDDYVKRGAAHTREVYATAAKVLGGTDVVFNDEFAPNNHITGSTIMGADARDSVVDKDCRTFDHPNLFISSSATMPTVGTVNVTLTIAALALRMSDTLKKEV

SEQ ID NO: 4 - protein, 425 aa, Burkholderia cepacia
VRKSTLTFLIAGCLALPGFARAADAADPALVKRGEYLATAGDCMACHTVKGGKPYAGGLGMPVPMLGKIYTSNITPDPDTGIGKWTFEDFERAVRHGVSKNGDNLYPAMPYVSYAKITDDDVRALYAYFMHGVEPVKQAPPKNEIPALLSMRWPLKIWNWLFLKDGPYQPKPSQSAEWNRGAYLVQGLAHCSTCHTPRGIAMQEKSLDETGGSFLAGSVLAGWDGYNITSDPNAGIGSWTQQQLVQYLRTGSVPGVAQAAGPMAEAVEHSFSKMTEADIGAIATYVRTVPAVADSNAKQPRSSWGKPAEDGLKLRGVALASSGIDPARLYLGNCATCHQMQGKGTPDGYYPSLFHNSTVGASNPSNLVQVILNGVQRKIGSEDIGMPAFRYDLNDAQIAALTNYVTAQFGNPAAKVTEQDVAKLR

SEQ ID NO: 5 - DNA, 3399 bp, Ewingella americana
  source 1..3399: mol_type = genomic DNA
  CDS 1..507: protein_id = 6; translation = MSDTPSKTLRVSGVSRRRLLGYIGVGLASSLMMPVPLKAFAADG EPSPVDVDKFLRISRALTGKKHLNPVIGQRLYAAILQRKPDIAAQLDSLQTLPAGAPS SWSQPQQDAARIILQGWYLGQVGEGQDIAVVTYEKALMFEAVSDVLVIRSYCTNRPGY WAAKPDVAL
  CDS 521..2131: protein_id = 7; translation = MNMADTLKADVVVIGSGVAGGLVANELAKAGKNVLILEAGPRLS RWEIVENFRNQADKSDNMKPYPSTDYAPHPEANPSNNYLIQKGEHPYDAQFIRVVGGT TWHWAASAWRFLPNDFKIKSLYGVGRDWPMEYGELEKWYFRAEQELGVWGPNNEELGS PRSAPYPMEPLPLSYNEQRLKTVLNNNGYYVVTEPVARNSRPYDGRPTCCGNNNCMPI CPIGAMYNGITHVEKAEKNGAVLRPESVVYKLEVNDKQEITAALFKDPQGNEHRAEAK YFVLAANGIEIPKLMLMSTNDKNPNGVGNSSDQVGRNLMDHPGVGVEFYANEPLWPGR GPQEMTSMVGFRDGDFRKEYAAKKIHLSNVSRTDQEAGRLLSQKGELILGPDLDKRIR DRSSRYVRFDSFHEILPHAENRIIPSTTEKDKLGIPKPEFYYAMDDYVRKSAEHTYKV YADAARLMGGTDITYFKDFANNNHITGTTIMGHDPKDSVVDTDCRTHDHKNLFIASSA VMPTVGSVNCTLTIAALSLRIADTLKAEV
  CDS 2137..3396: protein_id = 8; translation = MKKLTLSLMAAVGLFSLAAQAEESGVDLIKRGEYLTQAADCVAC HTTKDGKPFAGGLAFKTPMGTLYSPNITADKETGIGTWSDEDFLRAVHEGKNKEGQHL YPAFPYTSYTLLKDDDVKAIKAYLFSLPPVHQQNRENEMPFPFNQRWGMWFWNLVNFD SKRFEPDTAQSAEWNQGKYLVEGLGHCGECHTPRNITMGMKDGKPYAGTEVDGWTAFN ISSDKKAGIGSWSKGDIVQYLRSGHVSGKAQAAGPMAEVIENSTRHLTDADLNAMATY IQTLKPQADSSVTKSRSDWGHESTSVDNLRGVPFKASQETDGARLFLGNCASCHSFGG QGSQDGYYPSLMKNSVVGAQTPNNLVNVILHGVSRKTNDGEVFMPGFADTLSDKQIAT LSTYMFKEFGAQPVLNVKDKDVKDLRGE
atgtctgataccccttcgaagacgcttcgcgtcagcggcgtttcacgtcgtcgcttattgggttatatcggcgtcggactggccagcagcctgatgatgcccgttccgcttaaagcgtttgccgccgacggagaaccttcgccggttgatgtcgataaattcttgcgcatttcccgcgcgctaactggcaaaaagcacctcaacccggtgattggtcaacgcctgtatgctgcgattttgcagcgcaaacctgacatcgccgcgcagttggactcactacaaaccctgcctgccggtgcgccgtcgagctggtctcagcctcagcaggacgccgcgcgcatcatcctgcaaggctggtatttgggtcaggtgggtgaggggcaagacattgcagtcgtgacttatgaaaaagcgctgatgtttgaggcggtctccgatgtgctggtgatccgctcttactgtaccaatcgtcctggctactgggctgccaagcccgatgtcgcgctttaattaaggaaaaatgaacatggctgacacattaaaagctgacgtcgttgtcattggctcaggcgttgccggtggtttagtggcaaacgaactggcgaaggcggggaaaaacgtcttaattttggaagcaggcccgcgcctgtctcgctgggaaatcgtcgagaatttccgcaatcaggccgacaagtcggacaacatgaagccgtacccgtctaccgactacgcgccgcatcctgaagcgaacccaagcaataactatctgattcagaagggtgagcacccgtacgacgcccagtttatccgcgtggtgggtggcacaacctggcactgggcggcctccgcgtggcgcttcctgccgaacgacttcaagattaaatctctgtacggcgtaggccgcgactggccgatggagtacggcgagctggagaaatggtacttccgcgccgagcaagagctgggcgtgtgggggccaaacaatgaagagctgggttcgccgcgcagtgcgccatacccgatggaacctctgccgctctcttataacgaacagcgcctgaaaaccgtgctcaacaacaacggttactacgtggtgaccgagccggtggcgcgtaacagccgcccttatgatggccgcccgacgtgctgtggtaacaacaactgcatgccgatttgcccgattggcgcgatgtacaacggcatcacgcacgttgagaaagctgagaaaaacggcgccgtgctgcgccctgagtcggtggtgtacaagctggaagtcaacgacaagcaggagatcaccgccgcgctgttcaaagacccgcagggcaatgagcaccgcgccgaagccaaatacttcgtgctggcggcgaacggcattgaaatccctaaactgatgctgatgtccaccaacgacaagaaccctaacggcgtgggtaacagctctgaccaagtgggccgtaacttgatggaccatccgggcgtcggcgttgagttctacgccaacgagccgctgtggccggggcgcgggccgcaggagatgacctctatggttggcttccgcgacggcgatttccgtaaagagtacgccgcgaagaaaatccacctgtcgaacgtttcccgtaccgatcaggaagcagggcgtctgctcagccaaaaaggtgagttgatccttgggccagacctcgacaagcgcattcgtgaccgttcgtcgcgctacgtgcgttttgacagcttccacgagatcttgccgcacgcggaaaaccgcatcattccgagtaccacggagaaagacaaactgggcattccgaagccggagttctactacgcgatggatgactacgtgcgtaaaagtgccgagcacacttacaaagtctatgccgatgcggctcgcctgatgggcggcaccgacatcacttacttcaaagacttcgccaacaacaaccacattaccggcaccaccatcatgggccacgacccgaaagactcggtggtagataccgattgccgtactcacgaccacaaaaacctgtttatcgccagcagcgcggtcatgccgactgtcggctcggtaaactgtactttgactatcgcggcgctctcgctgcggattgcagacacgttaaaggcggaggtgtaaccatgaaaaagttaacccttagcctgatggctgctgttggtctgtttagcttggcggcgcaggccgaagagtctggcgtggacctgattaagcgcggtgaatacctgacgcaggccgcggactgcgtggcgtgtcacaccactaaagacggcaaaccttttgccggcggactggcttttaagacgccaatgggcacgctctactcgccaaacatcacggcggataaagagacgggcattggcacttggagcgacgaggacttcctgcgcgcggttcacgaaggtaagaacaaagagggccagcacctctatccggccttcccgtatacctcttacacgctgttgaaagatgacgacgtgaaggctatcaaggcttacttgttcagcctgccgccggtgcatcagcaaaatcgtgaaaacgagatgccgttcccgttcaaccagcgctggggcatgtggttctggaatctggtgaatttcgacagcaagcgttttgagccagacaccgcgcagtctgccgagtggaatcagggtaaatatctggtcgaaggcttaggccactgcggcgagtgccataccccgcgtaacatcacgatggggatgaaagacggcaagccgtacgccggaaccgaggttgacggctggaccgcgtttaacatctcttccgacaagaaagccgggattggtagctggagcaaaggcgacatcgtgcagtatctgcgttctggtcacgtcagtggcaaagcgcaggccgctggcccgatggcggaagttatcgagaacagtacccgccatctgaccgatgccgacctgaacgcgatggcgacctacatccagacgctgaagccgcaggcagattcttccgtcaccaagagccgttctgattggggccatgagagcaccagcgtagacaacctgcgcggcgtgccattcaaagccagccaagaaaccgacggcgcgcgtttgttcctcggcaactgcgcaagctgccacagcttcggcggtcagggcagccaagacggttactacccatcgctgatgaaaaacagcgtggtgggtgcccagacaccaaacaacttggtcaacgttatcctgcacggcgtatcgcgcaagaccaacgacggcgaagtgttcatgccgggctttgccgacaccctgagcgacaagcagatcgccacgctgagcacctatatgtttaaagagtttggcgcccagccggtgttgaatgtgaaagataaagatgtgaaggatttgcgcggggagtga

SEQ ID NO: 6 - protein, 169 aa, Ewingella americana
MSDTPSKTLRVSGVSRRRLLGYIGVGLASSLMMPVPLKAFAADGEPSPVDVDKFLRISRALTGKKHLNPVIGQRLYAAILQRKPDIAAQLDSLQTLPAGAPSSWSQPQQDAARIILQGWYLGQVGEGQDIAVVTYEKALMFEAVSDVLVIRSYCTNRPGYWAAKPDVAL

SEQ ID NO: 7 - protein, 537 aa, Ewingella americana
MNMADTLKADVVVIGSGVAGGLVANELAKAGKNVLILEAGPRLSRWEIVENFRNQADKSDNMKPYPSTDYAPHPEANPSNNYLIQKGEHPYDAQFIRVVGGTTWHWAASAWRFLPNDFKIKSLYGVGRDWPMEYGELEKWYFRAEQELGVWGPNNEELGSPRSAPYPMEPLPLSYNEQRLKTVLNNNGYYVVTEPVARNSRPYDGRPTCCGNNNCMPICPIGAMYNGITHVEKAEKNGAVLRPESVVYKLEVNDKQEITAALFKDPQGNEHRAEAKYFVLAANGIEIPKLMLMSTNDKNPNGVGNSSDQVGRNLMDHPGVGVEFYANEPLWPGRGPQEMTSMVGFRDGDFRKEYAAKKIHLSNVSRTDQEAGRLLSQKGELILGPDLDKRIRDRSSRYVRFDSFHEILPHAENRIIPSTTEKDKLGIPKPEFYYAMDDYVRKSAEHTYKVYADAARLMGGTDITYFKDFANNNHITGTTIMGHDPKDSVVDTDCRTHDHKNLFIASSAVMPTVGSVNCTLTIAALSLRIADTLKAEV

SEQ ID NO: 8 - protein, 420 aa, Ewingella americana
MKKLTLSLMAAVGLFSLAAQAEESGVDLIKRGEYLTQAADCVACHTTKDGKPFAGGLAFKTPMGTLYSPNITADKETGIGTWSDEDFLRAVHEGKNKEGQHLYPAFPYTSYTLLKDDDVKAIKAYLFSLPPVHQQNRENEMPFPFNQRWGMWFWNLVNFDSKRFEPDTAQSAEWNQGKYLVEGLGHCGECHTPRNITMGMKDGKPYAGTEVDGWTAFNISSDKKAGIGSWSKGDIVQYLRSGHVSGKAQAAGPMAEVIENSTRHLTDADLNAMATYIQTLKPQADSSVTKSRSDWGHESTSVDNLRGVPFKASQETDGARLFLGNCASCHSFGGQGSQDGYYPSLMKNSVVGAQTPNNLVNVILHGVSRKTNDGEVFMPGFADTLSDKQIATLSTYMFKEFGAQPVLNVKDKDVKDLRGE

SEQ ID NO: 9 - DNA, 3638 bp, Silvimonas terrae
  source 1..3638: mol_type = genomic DNA
  CDS 1..537: protein_id = 10; translation = MNDRDSPIHADALLPQPTRRRLLLASGLLLGTLAVSGRLVSVVF ADTPAQDSQFLQLSQLLTGRAQLDTAVAHRFYSALSTKDNQLDSKLRQIAGEAQTNSL TNAEELLTALDANQPELAKTARNIIAAWYSGVVGDGPDAIVITYASALMFDLVKDATM PPSYCQAAPLYWSAKPPLN
  CDS 555..2174: protein_id = 11; translation = MSANNQSADVIVIGSGVAGALVAHDLSAAGASVLMLEAGPRLER WRIVENYRNGAAKDDNQRPYPAARHAPHPEYGPDNNYLILKGPDAQSYASQYLRVVGG TTWHWAASCWRFLPNDFHMKQVYGVGRDWGISYADLEPWYYKAELELGVSGPNDGTDL GSPRKQPYPMDHLPLSYNDQRFHDVLNANGFNLVAEPVARNSRPYDERPTCCGNNNCM PICPIAAMYSGIIHVLKAERAGTRLLTNAVVYRLEVDAHDNIIAALYKDPDGNSHRVT GKYFVLAANALESAKILLMSTTDKFASGLANSSDQVGRNLMDHPGTGITFLANEPLWP GRGPMELTSIVDHRDGDFRREWASKKLHLNNQSQTRKATEKALSMGLVGKKLDEEIRY RAAHTVNINSFHDILPEPDNRLVLSDQKDPLGLPRMEITYKIGDYTKRSAEHTHESYA KIAQLFGGTEVTFDDKFAPNNHLMGSVIMGQNPKDSVVDGDCRAHDHANLYLATSGVM ASAGSVNCTLTIAALSLRIADTLKRQLGRTTS
  CDS 2193..3635: protein_id = 12; translation = MTCRLLRPFALAALMLVSALAQAAEVSPEQIKRGEYLARAADCI ACHTVPRTGQPFAGGYGIASPLGTIYSSNITPSTSAGIGNYTLQDFDNALRRGIRKDG AHLYPAMPYTAYAKLQPEDVEALYAYLMHGVKPVDAQPRRTRLPFPFNIRLSMAVWNS LFLDPRVFQPEQGKSEDFNRGKYLVEALEHCSACHTPRNMLMAENPDAPFAGGQLGAW YAPNITADPVSGVGGWTQAELVQYLKTGHVAGKAAAAGPMAEAVSDSLSHLTDPDLQA MATYLKTIAPIRNTADSKPAWAWGSPANDDDSLRGTAGVTQSNRPTSAAELFSANCAS CHQANGMGTTDTAYPSLTNNTALGHLNPSNLIAVILNGVDRPLPDGHHAFMPGFGPGS LINPLSDADIATLANYTLQRFGNTNVHVTPEEVAASRKGGEPPLLAKLPRVLPVVLML FALLLILGGWRSARQRKLRHVARPSPTPRTP
atgaatgatcgtgattcgcccattcacgccgatgcgctactcccccagcccacccgccgccgtctgttgctggcatctggcttgctgctggggacgctggccgtttccgggcgactggtctcggtggtctttgccgatacgcctgcgcaagacagccagtttttgcagctctcgcaattactgaccgggcgggcgcaactggataccgccgtcgcacaccgtttttattcagcgctgagcaccaaagacaatcagctggacagcaagctgcggcagattgcgggcgaagcgcaaaccaacagcctgaccaatgccgaagaattgctcaccgcgctggatgccaaccagccagaactggccaagaccgcgcgcaacatcattgccgcgtggtattccggtgtggtgggtgacggcccggatgccattgtgattacttatgccagcgccttgatgttcgatctggtcaaagacgccaccatgccgccctcttactgccaggccgcaccgctgtactggtcggccaaaccaccgctgaactgacccggaccctcttcatgagcgcaaacaatcaatctgcggatgtcattgtcattggttcaggggtagccggtgcgctggtggcgcatgatctctctgccgccggggcgtcagtgctgatgctggaagccggcccccggctggaacgctggcgcattgtcgaaaactaccgcaatggcgccgccaaagatgataaccagcgcccctacccggccgccagacacgcgccgcacccggaatacgggcccgacaacaactatctgatcctcaaaggcccggatgcgcagtcttatgcctcgcaatatctgcgggtcgttggcggcaccacctggcactgggccgcgtcctgttggcgctttctgcccaatgattttcatatgaagcaggtctacggcgtgggccgggactgggggatcagctatgccgatctggaaccctggtactacaaggccgagctggaactcggggtttccgggcccaacgacggcacagacctgggctcaccgcgcaagcagccctacccgatggatcacctgccgctgtcttacaacgaccagcggttccacgatgtcctcaacgccaacgggttcaatctggtggccgaaccggtggcccgcaacagccggccctacgacgagcgcccgacgtgctgtggcaacaacaactgcatgcccatctgcccgattgccgccatgtacagcggcatcatccatgtgctcaaagccgaacgcgcgggtacgcgattgctgaccaatgcggtggtgtaccggctggaagtagacgctcacgacaacatcattgctgcgctctacaaagacccggacggcaacagtcatcgggtcaccggcaagtatttcgtgctggcggccaatgccctggaatcagccaaaatcctgttgatgtcgaccaccgacaaatttgccagtggcctggccaacagttcagaccaggtaggccgtaatctgatggatcaccccggcactggcataacctttctggccaatgagccgctctggccgggccgcgggccgatggagctgacctcgattgtcgatcaccgcgacggggatttccggcgggagtgggccagcaagaagttgcacctcaataaccagtcccagacgcgcaaggccacagaaaaagccttgtcgatggggctggtgggcaagaagctggatgaagaaatccgctaccgggccgcgcatacggtcaacatcaacagttttcatgacattttgccggagccggacaaccgcctcgtcctgtcagaccagaaagacccgctgggcctgccccgcatggaaatcacctacaagattggggattacaccaagcgcagcgccgaacatacgcatgagtcctacgcaaaaattgcccagttgtttggcgggaccgaggtgacgtttgatgacaaatttgcccccaacaatcacctgatgggctcggtcatcatgggacaaaaccccaaagactcggtggtcgatggcgattgtcgcgcgcacgaccacgccaatctgtatctggctaccagcggggtgatggcgtcggccggcagtgtgaactgcacgctgaccatcgccgcgctgtccttgcgtattgccgacacgctcaagcgccagcttgggcgcaccaccagttaagacgggagcgctaccatgacctgccgtcttcttcgcccctttgcccttgcggccctgatgcttgtttctgcgctggcgcaggctgctgaggtcagcccggaacagatcaagcgcggcgagtatctggcccgcgccgccgactgtattgcctgtcataccgtgccgcgtaccggccagccgtttgcgggtggttacggcattgcctcgccgttgggcactatttattcgtccaacatcacgccgtccacttcggccgggattggcaactacacgctacaagatttcgacaacgccttgcgccggggcatccgcaaagatggcgcgcacctctacccggccatgccctacaccgcctacgccaagctgcagccagaagacgtcgaggcgctctatgcctatctgatgcatggcgtgaaaccggtagatgcccagccacgtcgcacgcggttgccgtttccgttcaacatccggctctccatggccgtctggaacagcttgtttctggacccacgggttttccagccagaacaaggcaagagtgaggacttcaatcgcggcaagtatctggtagaagcactggaacattgcagcgcctgccacacgccccgcaacatgctgatggcagaaaacccggacgcgccatttgccggcgggcaactgggtgcatggtatgcgcccaatatcaccgccgaccccgtcagcggcgttggcggctggacccaggcggaactggtgcaatacctcaaaaccggccatgtcgcgggcaaagctgcggccgccggcccgatggccgaagcggtctctgacagcttaagccatctgaccgacccagacctgcaggccatggcgacctacctcaaaaccatcgcccccatccgcaataccgccgacagcaaacccgcctgggcatggggtagcccggccaatgacgacgatagcttgcgcggtacggcgggcgtcacccaaagtaaccgcccaaccagcgcggccgaattgttcagcgccaattgcgccagttgccatcaagccaatggcatgggcacgaccgacaccgcctacccatcgctcaccaacaacacggcgttgggacaccttaaccccagcaacctgatcgccgtaatcctgaacggggtagaccggccactgccggacggccaccacgccttcatgcctggttttggccccggcagcctgatcaaccccttgtcggatgcggatatcgccacgctggccaattacacgttgcaacggttcggcaacaccaacgtccacgtcacgcccgaggaggtcgctgcctcgcgaaaaggcggtgagccgccactgctcgccaaattgccccgtgtattgccggtcgtgctgatgctgtttgcgctgttgctgattctgggcggctggcgcagtgcgcgacagcgcaaattgcgccatgtagcgcgcccctctcccacgccacgcacgccttga

SEQ ID NO: 10 - protein, 179 aa, Silvimonas terrae
MNDRDSPIHADALLPQPTRRRLLLASGLLLGTLAVSGRLVSVVFADTPAQDSQFLQLSQLLTGRAQLDTAVAHRFYSALSTKDNQLDSKLRQIAGEAQTNSLTNAEELLTALDANQPELAKTARNIIAAWYSGVVGDGPDAIVITYASALMFDLVKDATMPPSYCQAAPLYWSAKPPLN

SEQ ID NO: 11 - protein, 540 aa, Silvimonas terrae
MSANNQSADVIVIGSGVAGALVAHDLSAAGASVLMLEAGPRLERWRIVENYRNGAAKDDNQRPYPAARHAPHPEYGPDNNYLILKGPDAQSYASQYLRVVGGTTWHWAASCWRFLPNDFHMKQVYGVGRDWGISYADLEPWYYKAELELGVSGPNDGTDLGSPRKQPYPMDHLPLSYNDQRFHDVLNANGFNLVAEPVARNSRPYDERPTCCGNNNCMPICPIAAMYSGIIHVLKAERAGTRLLTNAVVYRLEVDAHDNIIAALYKDPDGNSHRVTGKYFVLAANALESAKILLMSTTDKFASGLANSSDQVGRNLMDHPGTGITFLANEPLWPGRGPMELTSIVDHRDGDFRREWASKKLHLNNQSQTRKATEKALSMGLVGKKLDEEIRYRAAHTVNINSFHDILPEPDNRLVLSDQKDPLGLPRMEITYKIGDYTKRSAEHTHESYAKIAQLFGGTEVTFDDKFAPNNHLMGSVIMGQNPKDSVVDGDCRAHDHANLYLATSGVMASAGSVNCTLTIAALSLRIADTLKRQLGRTTS

SEQ ID NO: 12 - protein, 481 aa, Silvimonas terrae
MTCRLLRPFALAALMLVSALAQAAEVSPEQIKRGEYLARAADCIACHTVPRTGQPFAGGYGIASPLGTIYSSNITPSTSAGIGNYTLQDFDNALRRGIRKDGAHLYPAMPYTAYAKLQPEDVEALYAYLMHGVKPVDAQPRRTRLPFPFNIRLSMAVWNSLFLDPRVFQPEQGKSEDFNRGKYLVEALEHCSACHTPRNMLMAENPDAPFAGGQLGAWYAPNITADPVSGVGGWTQAELVQYLKTGHVAGKAAAAGPMAEAVSDSLSHLTDPDLQAMATYLKTIAPIRNTADSKPAWAWGSPANDDDSLRGTAGVTQSNRPTSAAELFSANCASCHQANGMGTTDTAYPSLTNNTALGHLNPSNLIAVILNGVDRPLPDGHHAFMPGFGPGSLINPLSDADIATLANYTLQRFGNTNVHVTPEEVAASRKGGEPPLLAKLPRVLPVVLMLFALLLILGGWRSARQRKLRHVARPSPTPRTP

SEQ ID NO: 13 - DNA, 3500 bp, Zymobacter palmae
  source 1..3500: mol_type = genomic DNA
  CDS 1..585: protein_id = 14; translation = MSGPAIFPAVFNRRSEITVKDDTPLTDAGAVAPAFSQPVGVSRR CLLGGAALGLAAITLGALPVAQAADNAQDSALADFIRVSGVLTARQQLNPVLSGLIFG EMKKEDPAFADQLSSLAGVIHQPPAQWSASMTTLAREITSVWYTGLIGSGDTMRVVTY EGALQFSATKDIFIVRSYCPNRPGFWASQPSGWRV
  CDS 592..2193: protein_id = 16; translation = MAGIQKADVVIVGTGVAGALIAHQLAKAGKRVLMLEAGPRLTRG EITGNFRSSAFKSDFQSPYPCTDYAPIPTMHPKNDYFIQSGEHSYDAQYLRLVGGTTW HWAASAWRYLPVDFKLKSTYGVGVDWPIDYDALEPWYQRAEEALGVWGPNDEELGSPR SQPYPMTPLPLSYNEHTIKTRLNAQGFGIVTEPVARNSRPYDDRPTCCGNNNCMPICP IGAMYGGIVHVEKAEKAGAKVVPEAVVYRIETGDDNRIVAVHYKDPKGESHRVEGKYF VIAANGIETPKLLQISVDSRNPNGVANSSGWVGRHLMDHPGIGVRFYAKEPLWPGRGP QEMTSAVAWRDGDFRRTAAGKKIHLSNLSRVDQVTQELLAGDQLIFGNALEEQIRDRA ARFVQFDSFHEILPLPENRIVPSKVHTDKLGIPKPEFFYRIDDYVKRGAAHTREMYAR AAQVMGGTDIQYADDFANNQHICGTVMMGPTAETGVVDQHCRTFDHENLFLATSGVMP TVGTVNCTLTVAALSLRMAEVIKAEV
  CDS 2199..3497: protein_id = 15; translation = MASNFWRVSLGVGAMAALWMSGVAAAEDASTRQAELVKRGEYVA RTADCVACHTTDHDKPFAGGLPMGLPMGNIYSTNITPDKDTGIGSYTLEEFKRVLREG VTPDRGNLYPAMPYPSYTKMTDDEIEALYAYFMHGVQPIHQENKAPDFYWPLTIRWPL KIWNWMFLDEGAYQPKAGQSDEWNRGAYLVQGPAHCGTCHTPRGLAMQERAYDETGDE YLAGADIASWHAFNITSDINSGIGGWSADELVQYLKDGNVRGKGQAGGQMGEAVEHSF SHMTDADLHAIAVYMKTVPAVNDGDTRPRYSYGAPGDDFLRLRAQPVAQDAASDRGAR VYLENCAACHGPSGQGSPDGYYPSMFHNSVIGTDYHNNLINVVLDGVKRKTPGNDVLM PAFRDELSDEDIAALANYLSQQFGRGDMNVTPKSVKALRND
atgagtgggccggccatattccccgctgtgttcaatcgacgaagcgagatcactgtgaaagatgacaccccccttacggatgctggtgcggtggcgcctgctttctctcagcctgtcggcgtatcgcgtcgctgcctattgggtggtgccgcgctgggactggcagcgattacgctaggtgcgcttcccgtagcgcaggcagcagacaacgcccaagatagcgcgctggctgacttcatccgcgtatcaggtgtgctgacagcacgtcagcaactcaacccggtgctgtcaggactcatcttcggtgaaatgaagaaagaagatccggcctttgctgatcagctgtcttccttagcgggcgttatccatcagccgccagcgcagtggagtgcttccatgacgacgctggcgcgtgaaataacctccgtctggtacaccgggttgatcgggagcggagacaccatgcgtgtcgtgacgtatgaaggtgcgctccagttttcggcgacgaaggatatcttcattgtgcgcagctattgccccaatcgtcccggtttctgggccagccaaccttcaggatggagagtgtgattcatggcaggaattcaaaaggctgacgttgtcatcgttggcacaggggtggccggtgctctgattgcacatcagctggccaaggcgggcaagcgtgtactgatgctggaagcggggccgcgtctgacacgtggtgaaattactggcaactttcgttccagtgcgttcaagtcggattttcaatcgccatacccgtgtaccgactatgcgccgattccgacgatgcaccccaagaacgactacttcatccaaagcggtgagcactcctacgatgcgcagtacctgcgcttggttggtggtacgacttggcactgggcggcatcggcgtggcgctatctgccggtcgatttcaagctgaagagcacctatggtgtcggggttgactggcccatcgactacgatgcactggagccgtggtatcagcgtgctgaagaagcactgggtgtgtgggggcctaatgatgaagagctaggttcaccgcgttcccagccttatccaatgacgccgctgccgctgtcctataacgaacacacgatcaagacgcgcctcaatgcgcaggggttcgggatcgtgacggagccggtggcgcgcaatagccgcccctacgatgatcgccccacctgctgcggtaataacaactgcatgccgatctgccctattggcgcgatgtatggcggtatcgtccatgtcgaaaaagccgaaaaggcgggggctaaagtcgttcccgaggcggtggtgtaccgcatcgagacgggcgatgacaaccgtatcgtggcggtgcattacaaggatccgaaaggcgagtcgcaccgcgttgaaggtaaatacttcgtcatcgcggcaaacggcatcgaaacgccgaagttgttgcagatttcggtcgatagccgcaatccgaatggtgtggccaacagctctggctgggtgggtcgccacctgatggaccacccggggatcggcgtacgcttctatgccaaggaaccgttgtggccgggccgtgggcctcaggaaatgacctctgcggttgcgtggcgcgatggggatttccgacgcacggcagcgggcaaaaagatccacctatccaacctgtcgcgtgtcgatcaggtcacccaagagctccttgccggtgatcagctgattttcggaaacgcgctggaagagcagatccgtgatcgcgcagcacgcttcgtgcagttcgacagtttccacgaaatactgccgcttcctgaaaaccgcattgtgccgagcaaggtgcatacggacaaactggggattcccaagcctgaatttttctaccgcatcgacgattacgttaagcggggggcagcgcatacccgcgagatgtatgcgcgtgcggcacaggtcatggggggtacggatattcagtatgccgatgactttgctaataaccagcatatctgcggtacggtgatgatggggccaacggccgaaactggggtagtggatcagcactgccgtacgttcgatcacgagaacctgtttttggccaccagtggcgtcatgccgacggtgggaacggtcaactgtacgctgactgtggcggcactgtcgctgcgcatggccgaagtcatcaaggcggaggtgtgatgatggcgagcaacttttggcgggtgtcgctgggtgtgggggcgatggcggccctgtggatgagcggtgtggcggcagcggaagatgctagtacgcgacaggccgagctggtgaaacgaggtgagtatgtggcgcggacggcggactgtgtggcatgccataccactgatcatgacaagccctttgcgggtggtttgccgatggggttgccgatgggcaacatttattcgaccaatatcacgccggataaggatactgggatcggcagctatacgttggaggagttcaagcgtgtgctgcgtgaaggggtgacgccggatcgaggcaacctttatccggcgatgccgtatccgtcttataccaagatgaccgatgatgaaatcgaggcgctgtatgcctatttcatgcatggggttcaaccgattcatcaggaaaacaaggcgccggacttttactggccgctgaccattcgctggcctttgaagatatggaactggatgtttcttgatgaaggtgcctatcagcccaaggccgggcagagcgatgaatggaaccggggtgcctatcttgtgcaggggcctgcgcactgtggcacgtgccatactccgcgcgggctggcaatgcaggagcgcgcttatgatgagacaggcgacgagtacttggcgggcgctgatatcgcgagctggcatgctttcaacatcacttcggacatcaacagtgggatcggcggctggagtgccgatgagctggtgcagtatctgaaggatggcaatgtgcgtggcaaggggcaggccggagggcagatgggtgaggcggttgaacacagcttcagtcatatgaccgatgcagatctgcatgctatcgcggtgtatatgaagacggtgcctgccgtcaacgatggcgatacccgtccacgctatagctatggcgctcctggcgatgacttcctgcgccttcgggcccagcccgttgctcaagatgccgcatcggatcgaggggcgcgcgtgtatctggagaactgcgcggcttgccacgggccttccggacaaggctcgcctgatggctattatccgtcgatgttccataacagcgtcatcggtaccgattaccacaacaacctcatcaacgtggtattggatggcgtgaagcgcaagaccccaggcaacgacgtgctgatgccagccttccgcgatgagctgtccgatgaggatatcgcggcgttggctaactacctcagccagcagttcgggcgtggcgatatgaatgtgacgcccaaatccgtcaaggcactgcgcaacgattga

SEQ ID NO: 14 - protein, 195 aa, Zymobacter palmae
MSGPAIFPAVFNRRSEITVKDDTPLTDAGAVAPAFSQPVGVSRRCLLGGAALGLAAITLGALPVAQAADNAQDSALADFIRVSGVLTARQQLNPVLSGLIFGEMKKEDPAFADQLSSLAGVIHQPPAQWSASMTTLAREITSVWYTGLIGSGDTMRVVTYEGALQFSATKDIFIVRSYCPNRPGFWASQPSGWRV

SEQ ID NO: 15 - protein, 433 aa, Zymobacter palmae
MASNFWRVSLGVGAMAALWMSGVAAAEDASTRQAELVKRGEYVARTADCVACHTTDHDKPFAGGLPMGLPMGNIYSTNITPDKDTGIGSYTLEEFKRVLREGVTPDRGNLYPAMPYPSYTKMTDDEIEALYAYFMHGVQPIHQENKAPDFYWPLTIRWPLKIWNWMFLDEGAYQPKAGQSDEWNRGAYLVQGPAHCGTCHTPRGLAMQERAYDETGDEYLAGADIASWHAFNITSDINSGIGGWSADELVQYLKDGNVRGKGQAGGQMGEAVEHSFSHMTDADLHAIAVYMKTVPAVNDGDTRPRYSYGAPGDDFLRLRAQPVAQDAASDRGARVYLENCAACHGPSGQGSPDGYYPSMFHNSVIGTDYHNNLINVVLDGVKRKTPGNDVLMPAFRDELSDEDIAALANYLSQQFGRGDMNVTPKSVKALRND

SEQ ID NO: 16 - protein, 534 aa, Zymobacter palmae
MAGIQKADVVIVGTGVAGALIAHQLAKAGKRVLMLEAGPRLTRGEITGNFRSSAFKSDFQSPYPCTDYAPIPTMHPKNDYFIQSGEHSYDAQYLRLVGGTTWHWAASAWRYLPVDFKLKSTYGVGVDWPIDYDALEPWYQRAEEALGVWGPNDEELGSPRSQPYPMTPLPLSYNEHTIKTRLNAQGFGIVTEPVARNSRPYDDRPTCCGNNNCMPICPIGAMYGGIVHVEKAEKAGAKVVPEAVVYRIETGDDNRIVAVHYKDPKGESHRVEGKYFVIAANGIETPKLLQISVDSRNPNGVANSSGWVGRHLMDHPGIGVRFYAKEPLWPGRGPQEMTSAVAWRDGDFRRTAAGKKIHLSNLSRVDQVTQELLAGDQLIFGNALEEQIRDRAARFVQFDSFHEILPLPENRIVPSKVHTDKLGIPKPEFFYRIDDYVKRGAAHTREMYARAAQVMGGTDIQYADDFANNQHICGTVMMGPTAETGVVDQHCRTFDHENLFLATSGVMPTVGTVNCTLTVAALSLRMAEVIKAEV

SEQ ID NO: 17 - DNA, 3938 bp, Cobetia sp.
  source 1..3938: mol_type = genomic DNA
  CDS 1..621: protein_id = 18; translation = MTPRENSVSDDVPMRQDGHHEASQVAEPTSPARRRLLKLGVSGL GLVALSSLSLSALAASATSSEASSGAASDDDSAFKAFMLLSAWLLSDKTLDKRLGERF FEALVRIPAPNVPGMGSLPALKNKLLALGEARDYLKEDDLDASEMQLVRRVLQAWMLG TVGTAIDDPAAEVIAYEYAAMYAGPRDVQIIRTYCPNQPGFWAQRPE
  CDS 752..2362: protein_id = 19; translation = MAAESHSHDADVIIVGSGVAGALIAHRLAEAGKSVIMLEAGPRM QRWQIVERFRNQADKMDFMGPYPASKAAPHPMLYGENANYLVQKGEQVYDAQYIRAVG GTTWHWAASAWRFLPSDFRLKSEYGVGLDWPISYDDLEGYYFRAEQALGVWGPNDVDL GSPRSADYPMAPLPLSWNEQRVSQRINPHGFDMVTEPVARNSRPYDDRPTCCGNNNCM PICPIGAMYSGIIHVEKAERAGAKLIDNAVVHHLESDASGKIREVRYLDPQGKEHRLS APKVVLAANGIETPKLMQMSVNAHTPNGVGNQNDMVGRHLMDHPGTGVSFLADEVLWA GRGPQEMTSIITWRDGDFRKEYASKKLHLSNMARTQQMTTEVLSETPLRLGADLQAQI DNRASRYVQLDSFHELLPEPSNRITPSKKRDALGLPRPEFRYAIDDYVKRSAVHTREQ YARIAQLMGGTDIAFRDEFSNNQHICGTTLMGDDPKKSVVDRDCRVHGHDNLFVASSG CMPTVGSVNCTLTIAALSLRIADQLEQEI
  CDS 2368..3933: protein_id = 20; translation = MGQPGRNAVNDESIAKKRLMVRPAHRHLGSAIRLAAFAGVSTLL LSGCGNGEDEHAKQVAADKAATSDPALVKQGLYMAQASDCAACHTTKDGEDYAGGLPF ETPVGEIFSTNITPDTEHGIGNYTLEEYTRVLREGEAADGHNLYPAMPFPSYARLTDD DIKALYAWNMHDVTPSSEPNRESEIPFPLNMRWPMGLWEEIFSPLEPWQDDEQKEADW NRGAYLVQGPGHCGSCHTERGLAFQEKALTGDEEGYLGGAMIEGWRAFNLTPDVKDGL GSWSEQDIVTYLSTGNLKGKAQAGGPMADVISHSMRHMTDEDLNAMAVYLKSLPALNG KGEQGEAVNDDDRKAEATAPSEKQADEKGSQDAPQVATRFNQGAPADDVLLLRGQPLD KRPDDKELIGQLTDATPGVRLYLGHCAMCHGADGAGTQDGYYPSLFHNSVTGSVYPDN LAQAILNGVQRESNGHSVFMPAFDSSLNDNELINLMDYLENRFGEGHDSQTLAAPDAR RQLLSDKLKDWRGE
atgaccccacgtgaaaatagtgtttcggatgatgtccccatgcgccaggatggtcatcatgaagcgtcccaagtcgctgagccgaccagcccggcacgtcgtcgtctgctcaagctgggtgtgtcggggctgggattggtggcgctttccagcctgtcactttctgcactggcggccagtgccacctcatcagaggcgtcatccggtgccgcgtctgatgatgactcagccttcaaggcgttcatgctcctgtcagcatggttactgagcgacaagacgctggataaacgtctgggtgagcgtttctttgaggcattggtgcgtatccctgcccccaatgtgccaggcatgggtagtttgcccgccctgaagaacaagttgctggcacttggcgaggcgcgtgactatctgaaggaagacgatctcgacgcctccgagatgcagttggtgcgacgtgtcctccaagcctggatgctgggcacggttggcactgcaattgatgatcccgcggcggaagtcatcgcctatgaatacgcggccatgtatgccggccctcgcgatgtgcagatcatccgcacctattgccctaaccaaccgggcttctgggcacagcgtcctgagtgatcatgcctcgtcagtcctgaccctgatgctgtccctgatacgctttttgaacttcctgacaacttgactcgggtacttcccgatcaagattcacgtctaaaaaatgttcctctgaggagaatcctgcatggctgcggaatcccattcgcatgatgccgatgtcatcatcgtcggttctggtgttgctggcgccttgattgcccaccggcttgccgaggcaggtaaatcagtcatcatgcttgaggctggccctcgcatgcagcgctggcagattgtggagcgcttccgtaatcaagccgacaagatggacttcatggggccttatccggcaagcaaggctgcgccacacccgatgctgtatggcgagaatgccaattacctcgtgcagaagggcgagcaggtctatgacgcgcaatacattcgcgccgtcggtggtactacttggcactgggcggcgtctgcatggcgcttcctgcctagcgatttccgcttgaaaagcgaatatggcgtcggactggattggccgatttcctatgatgacctggaaggctactacttccgtgccgagcaggcgctgggggtatggggcccgaatgatgttgacctcggcagcccgcgcagcgctgattatccgatggcaccgttgccgctgtcatggaatgagcagcgtgtcagtcagcggatcaatccgcatggctttgacatggtcaccgaacctgtcgcgcgcaacagtcgcccgtacgacgatcgcccgacctgctgtggcaacaacaactgcatgccgatctgtccgatcggggcgatgtactccggcatcatccatgtcgagaaggccgaacgtgcgggtgccaagttgatcgacaatgctgtggtgcaccatctggaaagcgatgccagcggcaagatccgtgaagtgcgctatctcgatccgcagggcaaggaacatcgcctgagcgcgcccaaggttgtgctggcggccaacggtatcgaaacgcccaagctgatgcagatgtcggtgaatgcacatacgcccaatggtgtcggcaatcagaatgacatggtggggcgtcacctgatggatcaccccggtaccggcgtcagcttcctggccgatgaagtgctgtgggcagggcgtgggcctcaggagatgacctcgatcatcacttggcgtgatggggacttccgcaaggagtacgcctccaagaagctgcacctctccaacatggcgcgtacacagcagatgacgacggaggtgttgtcggaaaccccgctgcgcctgggcgccgacctgcaggcccagattgacaatcgtgccagccgttatgtgcaactggatagcttccatgagttgttgccggagccgtccaatcgcatcacgccatcaaaaaaacgcgatgcgctcggcctgccgaggccagaattccgttatgctatcgatgattacgtcaagcgcagcgccgtgcatacccgagaacaatatgcacgtatcgctcagctgatgggaggcacggatatcgcgttccgtgacgaattctccaacaaccagcatatctgcggtacgaccctgatgggagacgaccccaagaagtccgtggtggaccgcgattgccgtgtgcatggccatgacaacctgtttgtcgccagctccggctgcatgccgaccgttgggtcggtcaattgcaccctgaccatcgcggccttgtctctccgtatcgccgatcagctagaacaggaaatatgagcatgggccagccaggtcgtaacgccgtgaatgatgagagtattgccaagaaacgcctgatggtccgtcctgcgcatcgtcatctgggaagcgccattcgtctagcggcatttgccggcgtcagcacactgctgttgagtggttgtggcaacggggaagatgagcacgccaagcaggttgctgctgacaaggcggccaccagtgacccggccttggtcaagcaaggtctttatatggcgcaggccagtgactgtgctgcctgccacacgaccaaggatggtgaagactacgcaggtggcctcccgttcgagacaccggtcggtgagattttctccaccaacatcacgccggacaccgagcatggcatcggcaactatacccttgaggaatacacccgtgtgctgcgcgaaggggaagcagctgacggccataacctgtacccggcgatgccgttcccgtcctacgcgcgtctgaccgatgatgacatcaaggcgctgtatgcgtggaacatgcatgatgtcacgccgagcagcgagccgaatcgtgagagcgaaattccgttcccgctcaacatgcgttggccgatgggcctgtgggaagaaatcttctcaccgcttgagccgtggcaggatgacgagcagaaagaggctgactggaatcgtggcgcctatctggtgcagggcccgggtcactgcggtagctgccataccgaacgcggccttgccttccaggagaaggcgctgaccggcgatgaagaagggtatctcggtggtgcgatgattgaaggctggcgtgccttcaacctgacgccggacgtcaaggatggcttgggtagctggagcgagcaggatatcgtcacctatctctcgacgggcaacttgaaaggcaaggcacaggcgggtggcccgatggcggatgtcatcagtcactccatgcgccatatgactgacgaagacctcaatgcgatggcggtctatctgaagtcactgcccgcattgaatggcaagggcgagcagggcgaggcggtcaatgatgatgaccgcaaggcggaggctacggcgccatctgagaagcaggccgacgagaagggctcgcaagatgccccgcaggtcgcgactcgcttcaatcagggcgcaccggccgatgacgtgctgttgcttcgtgggcagccgctggacaagcgtccggatgacaaggagttgatcggccagctgacggatgcaacgcctggcgtgcgtctgtatctgggccactgtgccatgtgtcacggtgctgatggtgcgggtacgcaagacggctactacccgtcactgttccacaacagtgtcaccggcagcgtctatccggataatctggcgcaggccattctcaatggcgtacagcgtgagagcaatggccacagtgtcttcatgccggcctttgacagcagcctgaacgacaatgagctgatcaatctgatggactatctggagaatcgcttcggagaaggccatgacagccagacactggccgcgccagatgcgcgtcgtcagctgctgagtgacaagctcaaggattggcgtggcgagtaagc

SEQ ID NO: 18 - protein, 207 aa, Cobetia sp.
MTPRENSVSDDVPMRQDGHHEASQVAEPTSPARRRLLKLGVSGLGLVALSSLSLSALAASATSSEASSGAASDDDSAFKAFMLLSAWLLSDKTLDKRLGERFFEALVRIPAPNVPGMGSLPALKNKLLALGEARDYLKEDDLDASEMQLVRRVLQAWMLGTVGTAIDDPAAEVIAYEYAAMYAGPRDVQIIRTYCPNQPGFWAQRPE

SEQ ID NO: 19 - protein, 537 aa, Cobetia sp.
MAAESHSHDADVIIVGSGVAGALIAHRLAEAGKSVIMLEAGPRMQRWQIVERFRNQADKMDFMGPYPASKAAPHPMLYGENANYLVQKGEQVYDAQYIRAVGGTTWHWAASAWRFLPSDFRLKSEYGVGLDWPISYDDLEGYYFRAEQALGVWGPNDVDLGSPRSADYPMAPLPLSWNEQRVSQRINPHGFDMVTEPVARNSRPYDDRPTCCGNNNCMPICPIGAMYSGIIHVEKAERAGAKLIDNAVVHHLESDASGKIREVRYLDPQGKEHRLSAPKVVLAANGIETPKLMQMSVNAHTPNGVGNQNDMVGRHLMDHPGTGVSFLADEVLWAGRGPQEMTSIITWRDGDFRKEYASKKLHLSNMARTQQMTTEVLSETPLRLGADLQAQIDNRASRYVQLDSFHELLPEPSNRITPSKKRDALGLPRPEFRYAIDDYVKRSAVHTREQYARIAQLMGGTDIAFRDEFSNNQHICGTTLMGDDPKKSVVDRDCRVHGHDNLFVASSGCMPTVGSVNCTLTIAALSLRIADQLEQEI

SEQ ID NO: 20 - protein, 522 aa, Cobetia sp.
MGQPGRNAVNDESIAKKRLMVRPAHRHLGSAIRLAAFAGVSTLLLSGCGNGEDEHAKQVAADKAATSDPALVKQGLYMAQASDCAACHTTKDGEDYAGGLPFETPVGEIFSTNITPDTEHGIGNYTLEEYTRVLREGEAADGHNLYPAMPFPSYARLTDDDIKALYAWNMHDVTPSSEPNRESEIPFPLNMRWPMGLWEEIFSPLEPWQDDEQKEADWNRGAYLVQGPGHCGSCHTERGLAFQEKALTGDEEGYLGGAMIEGWRAFNLTPDVKDGLGSWSEQDIVTYLSTGNLKGKAQAGGPMADVISHSMRHMTDEDLNAMAVYLKSLPALNGKGEQGEAVNDDDRKAEATAPSEKQADEKGSQDAPQVATRFNQGAPADDVLLLRGQPLDKRPDDKELIGQLTDATPGVRLYLGHCAMCHGADGAGTQDGYYPSLFHNSVTGSVYPDNLAQAILNGVQRESNGHSVFMPAFDSSLNDNELINLMDYLENRFGEGHDSQTLAAPDARRQLLSDKLKDWRGE

SEQ ID NO: 21 - protein, 12 aa, synthetic construct
HMGLTKIHYSAL

SEQ ID NO: 22 - DNA, 36 bp, synthetic construct
  source 1..36: mol_type = other DNA
cacatgggtctgaccaaaattcattatagcgcactg

SEQ ID NO: 23 - DNA, 26 bp, synthetic construct
  source 1..26: mol_type = other DNA
catgccatggcacacaacgacaacac

SEQ ID NO: 24 - DNA, 30 bp, synthetic construct
  source 1..30: mol_type = other DNA
gtcgacgatcttcttccagccgaacatcac

SEQ ID NO: 25 - protein, 17 aa, synthetic construct
VAKLRHMGLTKIHYSAL

SEQ ID NO: 26 - DNA, 59 bp, synthetic construct
  source 1..59: mol_type = other DNA
gtcgcgaagctgcgccacatgggtctgaccaaaattcattatagcgcactgtgaaagct

SEQ ID NO: 27 - protein, 60 aa, synthetic construct
MRKSTLTFLIAGCLALPGFARAADHMGLTKIHYSALLRGVAADHMGLTKIHYSALLRGVA

SEQ ID NO: 28 - DNA, 181 bp, synthetic construct
  source 1..181: mol_type = other DNA
atgcggaaatctactctcactttcctcatcgccggctgcctcgcgttgccgggcttcgcgacgcgcggccgatcacatgggtctgaccaaaattcattatagcgcactgctgcgcggtgtcgcggccgatcacatgggtctgaccaaaattcattatagcgcactgctgcgcggtgtcgcg

SEQ ID NO: 29 - protein, 20 aa, synthetic construct
ALADHMGLTKIHYSALNPGT

SEQ ID NO: 30 - DNA, 60 bp, synthetic construct
  source 1..60: mol_type = other DNA
gcgcttgcagaccacatgggtctgaccaaaattcattatagcgcactgaaccccggcact

SEQ ID NO: 31 - protein, 15 aa, synthetic construct
HMGLTKIHYSALADT

SEQ ID NO: 32 - DNA, 62 bp, synthetic construct
  source 1..62: mol_type = other DNA
gagaggcaagcctgatgcacatgggtctgaccaaaattcattatagcgcactggccgatacc

SEQ ID NO: 33 - protein, 16 aa, synthetic construct
ERQAHMGLTKIHYSAL

SEQ ID NO: 34 - DNA, 49 bp, synthetic construct
  source 1..49: mol_type = other DNA
gagaggcaagcccacatgggtctgaccaaaattcattatagcgcactga

SEQ ID NO: 35 - protein, 16 aa, synthetic construct
KKEVHMGLTKIHYSAL

SEQ ID NO: 36 - DNA, 62 bp, synthetic construct
  source 1..62: mol_type = other DNA
aagaaggaagtccacatgggtctgaccaaaattcattatagcgcactgtgaccatgcggaaa

(19) United States
(12) Patent Application Publication
(10) Pub. No.: US 2026/0242842 A1
(43) Pub. Date: Aug. 20, 2026

(54) ORIENTATION CONTROLLABLE DIRECT ELECTRON TRANSFER TYPE GLUCOSE DEHYDROGENASES AND THEIR BIOSENSOR APPLICATIONS

(71) Applicants: THE UNIVERSITY OF NORTH CAROLINA AT CHAPEL HILL, Chapel Hill, NC (US); ARKRAY, INC., Kyoto-shi, Kyoto (JP)

(72) Inventors: Koji SODE, Chapel Hill, NC (US); Junko SHIMAZAKI, Chapel Hill, NC (US); Thy LE, Chapel Hill, NC (US)

(73) Assignees: THE UNIVERSITY OF NORTH CAROLINA AT CHAPEL HILL, Chapel Hill, NC (US); ARKRAY, INC., Kyoto-shi, Kyoto (JP)

(21) Appl. No.: 19/489,518

(22) PCT Filed: May 31, 2024
(86) PCT No.: PCT/US2024/032020

§ 371 Date: Dec. 2, 2025

Related U.S. Application Data
(60) Provisional application No. 63/505,827, filed on Jun. 2, 2023.

(51) Int. Cl. C12Q 1/00; C12N 9/04

(52) U.S. Cl. CPC C12Q 1/006; C12N 9/0006; C07K 2319/70; C12Y 101/01118

(57) ABSTRACT

The present disclosure relates to a modified glucose dehydrogenase according to one embodiment of the invention comprises a catalytic subunit, an electron transfer subunit, and a hitchhiker subunit, wherein a carbon nanotube binding peptide (CNTBP) is fused to at least one of the catalytic subunit, the electron transfer subunit, and the hitchhiker subunit; an enzyme electrode comprising a carbon nanotube and the modified glucose dehydrogenase; and a biosensor comprising the enzyme electrode.

### FIELD OF THE INVENTION

The present invention provides glucose dehydrogenases (GDHs) harboring flavin adenine nucleotide (FAD; FADGDH) capable of direct electron transfer (DET) with electrode comprising: a catalytic subunit (α-subunit); an electron transfer subunit (β-subunit); and a hitchhiker subunit (γ-subunit); wherein N-terminal or C-terminal of each subunit is fused with a peptide sequences which show specific binding with carbon nanotube, thereby the orientation of FADGDH on CNT can be uniformly controllable. This invention also discloses FADGDH harboring a subunit which is fused with a peptide sequence which shows specific binding with carbon nanotube, and a glucose sensor comprises with the above FADGDH.

### BACKGROUND OF THE INVENTION

Glucose dehydrogenase (GDH) is an enzyme often used for glucose sensors. Glucose sensors for daily use in self-blood glucose measurement by diabetic patients are required to show accurate measured values in any situation or environment. Therefore, the enzyme, which is a key factor of the performance, is required to have high stability so as to avoid heat inactivation (J Diabetes Sci Technol. 2011 Sep. 1; 5 (5): 1068-76.).

We have been engaged in the engineering of bacterial GDH harboring flavin adenine nucleotide (FAD; FADGDH) capable of direct electron transfer (DET) with electrode, and its applications to construct biomedical devices such as sensors for blood glucose monitoring, sensors for continuous glucose monitoring, enzyme fuel cells for glucose energy scavenging system, and biocapacitor as the self-powered glucose sensing system (Current Opinion in Electrochemistry

### Volume 12, December 2018, Pages 92-100 and Bioelectrochemistry Volume 132, April 2020, 107414).

The FADGDH is comprised of the following three distinct subunits; a catalytic subunit; alpha (α)-subunit; an electron transfer subunit; beta (β)-subunit and a hitchhiker subunit; gamma (γ)-subunit.

The catalytic subunit harbors FAD as the cofactor and also iron-sulfur (Fe—S) cluster as the primary electron acceptor from FAD in the catalytic subunit. The electron transfer subunit is comprised of three heme binding regions (domains), and the first heme binding region works as the electron acceptor from catalytic subunit. The electron transfer subunit is synthesized intracellularly, and then is transport to periplasmic space and matured to form covalently bound hemes in the periplasmic space. The hitchhiker subunit is predicted to recognize and bind catalytic subunit during the synthesis of these molecule, intracellularly, and then the hitchhiker subunit-catalytic subunit complex is transferred to periplasmic space by TAT machinery, to form three subunit complex with the electron transfer subunit the in periplasmic space. There is one inherent disulfide bond between catalytic subunit and hitchhiker subunit. The presence of an electron-transfer subunit makes the FADGDH complex capable of DET with electrode. The former studies of our group elucidated that the FADGDH with electron transfer subunit which is truncated to comprise with the first heme binding region alone (truncated beta-subunit; trβ), showed DET GDH properties (US2019/0010215A1). This fact suggested that the first heme binding region interact with catalytic subunit to accept electron during the catalytic reaction of glucose oxidation.

We have also developed super stabilized FADGDH, by introducing inter-subunit disulfide bonds, thereby prevents the dissociation of each subunit from the hetero-tetrameric enzyme complex (US2022/0306996A1).

One of the challenging issues to develop enzyme sensors with DET-type oxidoreductases is the orientation control of enzyme on the surface of the electrode, where enzyme and electrode proceed redox reaction to generate current which provides the signal of the enzyme sensors. The most popular methods to immobilize DET-type enzymes are the physical adsorption together with the cross-linking using chemical cross linkers such as glutaraldehyde, or the use of self-assembled monolayer (SAM) with functional group to form covalent bound with enzyme. However, these methods are not able to control the orientation of DET-type enzymes. The randomly orientated DET-type enzymes on the surface of the electrode include diversified distances between redox center of enzymes and electrode. Ideally, the DET-type enzyme should be uniformly oriented with the shortest distance between redox center and electrode, thereby yields the maximum current density, which results the best accuracy and sensitivity of sensors. Therefore, technologies as well as the engineered DET-type enzymes, which are able to control the orientation of enzyme are expected to be developed.

### SUMMARY OF THE INVENTION

To achieve control over the orientation of the FADGDH enzyme, we plan to employ a strategy involving the fusion of carbon nanotube binding peptide (CNTBP) to FADGDH. These peptides, which can bind specifically to carbon nanotube (CNT) electrode surfaces, can serve as “bridges” that orient the enzyme in the desired configuration. Thus, FADGDH can be immobilized on the electrode without SAM and be controlled in its orientation with respect to the electrode.

The overarching goal of this study is to optimize the DET ability of FADGDH for bio-electrochemical applications. To achieve this, we aim to control the orientation of the immobilized enzyme on the electrode surface, as this is a key determinant of its DET efficiency. Specifically, we plan to fuse CNTBP to FADGDH to enhance its ability to autonomously orient itself ideally towards the electrode surface. This is expected to maximize the efficiency of electron transfer, leading to improved accuracy and sensitivity in glucose monitoring.

To fully realize the benefit of utilizing DET-type enzymes for bio-electrochemical applications, it is imperative to control the orientation of immobilized enzymes to optimize their DET ability towards the electrode surface. In this invention, we disclose our strategies in maximizing the DET ability of FAD GDH by engineering FADGDH to empower its ability to autonomously face the ideal orientation towards the electrode surface.

Our group has been engineering DET-type FADGDH, which is composed of a catalytic subunit (α-subunit), an electron transfer subunit harboring three hemes (β-subunit), and a small subunit for post-translational modification (γ-subunit). An engineered version of this enzyme, FADGDHtrβ, harbors the truncated β-subunit composed only with the heme binding motif necessary for intra- and inter-molecular electron transfer. By further engineering FADGDHtrβ, we investigated the method of immobilization. The method is to fuse carbon nanotube binding peptide (CNTBP) with FADGDHtrβ (CNTBP-FADGDHtrβ) to control the binding of FADGDHtrβ to the surface of carbon nanotube modified electrodes. By investigating multiple fusion sites, we discovered the ideal location for CNTBP fusion, maximizing FADGDHtrβ's DET-ability.

The present invention provides glucose dehydrogenases (GDHs) harboring flavin adenine nucleotide (FAD) capable of direct electron transfer (DET) with electrode comprising: a catalytic subunit (α-subunit); an electron transfer subunit (β-subunit); and a hitchhiker subunit (γ-subunit); wherein N-terminal or C-terminal of each subunit is fused with a peptide sequences which show specific binding with carbon nanotube, thereby the orientation of FADGDH on CNT can be uniformly controllable. This invention also discloses FADGDH capable of direct electron transfer (DET) with electrode comprising: a catalytic subunit (α-subunit); a truncated electron transfer subunit (trβ-subunit); and a hitchhiker subunit (γ-subunit); wherein N-terminal or C-terminal of each subunit is fused with a peptide sequences which show specific binding with carbon nanotube, thereby the orientation of FADGDH on CNT can be uniformly controllable.

This invention also discloses FADGDH harboring a truncated electron transfer subunit (trβ-subunit) wherein C-terminal of the subunit is fused with a peptide sequence which shows specific binding with carbon nanotube, thereby the orientation of FADGDH on CNT can be uniformly controllable. This invention also discloses FADGDH harboring a truncated electron transfer subunit (trβ-subunit) wherein C-terminal of the subunit is fused with a peptide sequence comprises

(SEQ ID NO: 21)
NH₂-HMGLTKIHYSAL-COOH

which shows specific binding with carbon nanotube, thereby the orientation of FADGDH on CNT can be uniformly controllable.

More specifically, this invention discloses FADGDH harboring a subunit which is fused with a peptide sequence which shows specific binding with carbon nanotube, and a glucose sensor comprises with the above FADGDH.

In one mode of the present invention, discloses FADGDH harboring a subunit which is fused with a peptide sequence which shows specific binding with carbon nanotube, and a glucose sensor comprises with the above FADGDH.

One aspect of the invention is to provide a modified glucose dehydrogenase comprising a catalytic subunit, an electron transfer subunit, and a hitchhiker subunit, wherein a carbon nanotube binding peptide is fused to at least one of the catalytic subunit, the electron transfer subunit, and the hitchhiker subunit. In one embodiment, said carbon nanotube binding peptide comprises the amino acid sequence of SEQ ID NO: 21.

In one embodiment, said electron transfer subunit is a truncated electron transfer subunit. In one embodiment, said carbon nanotube binding peptide is fused to said electron transfer subunit. In one embodiment, said carbon nanotube binding peptide is fused to N-terminal portion or C-terminal portion of at least one of the catalytic subunit, the electron transfer subunit, and the hitchhiker subunit. In one embodiment, said modified glucose dehydrogenase is from Burkholderia cepacia.

Another aspect of the invention is to provide an enzyme electrode comprising the modified glucose dehydrogenase as described above and carbon nanotube.

Another aspect of the invention is to provide a glucose sensor comprising the enzyme electrode as described above.

Another aspect of the invention is to provide a method of measuring glucose comprising measuring glucose by using the glucose sensor as described above.

In one embodiment, voltage of at least-0.2V with respect to Ag/AgCl electrode is applied to the enzyme electrode.

### BRIEF DESCRIPTION OF DRAWINGS

FIG. 1A shows the construction of CNTBP-FADGDH wherein CNTBP is fused to C-terminus of the truncated β-subunit of Burkholderia cepacia FADGDH (BcGDH) (FADGDH-γαtrβ_{C}-CNTBP).

FIG. 1B shows the construction of CNTBP-FADGDH wherein CNTBP is fused to N-terminus of the truncated β-subunit of Burkholderia cepacia FADGDH (BcGDH) (FADGDH-γαtrβ_{N}-CNTBP).

FIG. 1C shows the construction of CNTBP-FADGDH wherein CNTBP is fused to N-terminus of the Y-subunit of Burkholderia cepacia FADGDH (BcGDH) (FADGDH-γ_{N}-CNTBP-αtrβ).

FIG. 1D shows the construction of CNTBP-FADGDH wherein CNTBP is fused to N-terminus of the α-subunit of Burkholderia cepacia FADGDH (BcGDH) (FADGDH-γα_{N}-CNTBP-trβ).

FIG. 1E shows the construction of CNTBP-FADGDH wherein CNTBP is fused to C-terminus of the γ-subunit of Burkholderia cepacia FADGDH (BcGDH) (FADGDH-γ_{C}-CNTBP-αtrβ).

FIG. 1F shows the construction of CNTBP-FADGDH wherein CNTBP is fused to C-terminus of the α-subunit of Burkholderia cepacia FADGDH (BcGDH) (FADGDH-γα_{C}-CNTBP-trβ).

FIG. 2A shows the images of electrophoresis of the FADGDH-γαtrβ_{C}-CNTBP and FADGDH-γαtrβ_{N}-CNTBP. M: marker; S: supernatant fraction; AEC: after anion exchange chromatography; SEC: C4 and C5 fractions after size exclusion chromatography.

FIG. 2B shows the images of electrophoresis of the FADGDH-γα_{N}-CNTBP-trβ and FADGDH-γ_{N}-CNTBP-αtrβ. M: marker; Su: supernatant fraction; A: after anion exchange chromatography; S1 and S2: C4 and C5 fractions after size exclusion chromatography, respectively.

FIG. 3 shows the results of cyclic voltammetry using various CNTBP-FADGDHs.

FIG. 4 shows glucose concentration dependence of current values at each potential using FADGDH-γαtrβ_{C}-CNTBP, FADGDH-γαtrβ_{N}-CNTBP and FADGDH-γαtrβ.

### DETAILED DESCRIPTION

A modified glucose dehydrogenase according to one embodiment of the invention comprises a catalytic subunit, an electron transfer subunit, and a hitchhiker subunit, wherein a carbon nanotube binding peptide (CNTBP) is fused to at least one of the catalytic subunit, the electron transfer subunit, and the hitchhiker subunit.

Hereinafter, the modified glucose dehydrogenase may be referred to as a CNTBP-GDH, or CNTBP-FADGDH or CNTBP-BcGDH.

GDH is a trimeric enzyme composed of three subunits and uses FAD as a coenzyme (trimeric FADGDH). Examples of the trimeric FADGDH include FADGDHs from Burkholderia cepacia, Burkholderia cenocepacia, Burkholderia thailandensis, Ralstonia pickettii, Ralstonia solanacearum, Burkholderia phytofirmans, Ralstonia sp., Moraxellaceae bacterium, Pseudomonas knackmussii, Yersinia nurmii, Edaphovirga cremea, Silvimonas terrae, Zymobacter palmae, Cobetia sp., Halomonas sp. and Ewingella americana.

### <Catalytic Subunit>

The catalytic subunit, also called as alpha subunit, is a polypeptide having a FAD-dependent glucose dehydrogenase activity. The catalytic subunit may include Fe—S cluster.

An example of alpha subunit includes SEQ ID NO: 3 which is an amino acid sequence of the catalytic subunit from Burkholderia cepacia KS1 strain.

The Burkholderia cepacia KS1 strain has been deposited with International Patent Organism Depositary, National Institute of Advanced Industrial Science and Technology (currently Patent Microorganisms Depositary, Biological Resource Center, National Institute of Technology and Evaluation (NPMD)) as of Sep. 25, 2000 under the accession number FERM BP-7306.

As described above, FADGDHs are known from various microorganisms and their catalytic subunits have a function as a catalytic subunit and their amino acid sequences may have a sequence identity of not less than 60%, not less than 80%, not less than 90%, not less than 95%, or not less than 98% to the amino acid sequence of SEQ ID NO:3. The amino acid sequence identity herein can be defined by aligning two amino acids such that the number of matched amino acids is maximum while inserting a gap(s) when necessary, and calculating the ratio of the number of matched amino acids to the total number of amino acids in the aligned portion (the same applies hereinafter).

The function of the catalytic subunit means a function of forming a complex with the electron transfer subunit and the hitchhiker subunit and exhibiting glucose dehydrogenase activity.

Examples of the amino acid sequence of the GDH a subunit having an identity of not less than 60% to SEQ ID NO:3 include the sequence of the a subunit of each of the followings: Burkholderia cenocepacia J2315 strain (GenBank Accession No. WP_006482972), Burkholderia thailandensis TXDOH strain (WP_009900298), Ralstonia pickettii 12D strain (WP_012761508), Ralstonia solanacearum IPO1609 strain (WP_003265143), Burkholderia phytofirmans PsJN strain (WP_012428610), Ralstonia sp. (WP_048931828), Moraxellaceae bacterium (WP_114899537), Pseudomonas knackmussii (WP_043248521), Yersinia nurmii (WP_049596850), Edaphovirga cremea (WP_114193339), Silvimonas terrae (WP_184102398) (SEQ ID NO: 11), Zymobacter palmae (WP_027705356) (SEQ ID NO: 15), Cobetia sp. (WP_158773851) (SEQ ID NO: 19), Halomonas sp. (WP_107334716) and Ewingella americana (WP_034788281) (SEQ ID NO: 7).

The amino acid sequence of the catalytic subunit (a subunit) may contain not only the mutation that introduces cysteine for disulfide bond formation with β subunit (for example, P205C, US2022/0306996A1), but also an additional mutation. Known examples of mutants comprising mutations in the catalytic subunit of FADGDH derived from Burkholderia cepacia include a mutant in which the amino acid residues at positions 472 and 475 are substituted (WO 2005/103248), a mutant in which the amino acid residues at positions 326, 365, and 472 are substituted (QYY mutant JP 2012-090563 A), a mutant in which position 365 and position(s) 326, 472, 475, 529, and/or the like are substituted (WO 2006/137283). The catalytic subunit of the modified FADGDH according to one embodiment of the present invention may have these mutations.

### <Electron Transfer Subunit>

The electron transfer subunit is also called β subunit, and contains at least one heme-binding domain. The subunit has a function to transfer electrons generated by reaction using glucose as a substrate, to an electrode.

The electron transfer subunit is not limited as long as it has a function to transfer electrons generated by glucose dehydrogenation to an electrode. Electron transfer subunits derived from various organisms, including known electron transfer subunits, may be used. The electron transfer subunit may be derived from the same microorganism as the microorganism from which the catalytic subunit is derived. Examples of the electron transfer subunit include the electron transfer subunit of GDH of Burkholderia cepacia. Examples of the FADGDH electron transfer subunit of the B. cepacia KS1 strain include a polypeptide comprising the amino acid sequence of SEQ ID NO:4. SEQ ID NO:4 is an amino acid sequence of the FADGDH B subunit of the Burkholderia cepacia KS1 strain.

The electron transfer subunit contains three heme-binding domains. The electron transfer subunit may be a mutant (truncated) electron transfer subunit in which the first heme-binding domain and the second heme-binding domain, that is, the first and second domains as counted from the N-terminal side, respectively, are deleted. Such a truncated electron transfer subunit only containing the third heme-binding domain is disclosed in US 2019-0010215.

Thus, in the electron transfer subunit having three heme-binding domains (CXXCH), the first and second heme-binding domains as counted from the N-terminal side, or a region(s) including these, may be deleted. The following description uses the electron transfer subunit of Burkholderia cepacia as a representative example. In SEQ ID NO:4, there are the first heme-binding domain (amino acid positions 43 to 47), second heme-binding domain (amino acid positions 191 to 195), and third heme-binding domain (amino acid positions 334 to 338). Among these, the first heme-binding domain and second heme-binding domain may be deleted, or the region including the first and second heme-binding domains (amino acid positions 43 to 195) may be deleted.

Examples of a truncated electron transfer subunit in which the region including the first and second heme-binding domains is deleted include a truncated electron transfer subunit composed of amino acid positions 314 to 425 or 330 to 425 of SEQ ID NO:4.

Polypeptides having some degree of sequence identity to SEQ ID NO: 4 can be used for preparing an electron transfer subunit of CNTBP-GDH as long as they maintain the function of the electron transfer subunit. The function of the electron transfer subunit means a function of forming a complex with the catalytic subunit and the hitchhiker subunit and exhibiting the above-described electron transfer function.

For example, amino acid sequence of the electron transfer subunit may have a sequence identity of not less than 60%, not less than 80%, not less than 90%, not less than 95%, or not less than 98% to the amino acid sequence of SEQ ID NO:4. Preferably, three heme binding regions are conserved in the amino acid sequence of the electron transfer subunit.

Or in the case of a truncated form only comprising the third three heme-binding domain, amino acid sequence of the truncated electron transfer subunit may have a sequence identity of not less than 60%, not less than 80%, not less than 90%, not less than 95%, or not less than 98% to the amino acid sequence of amino acids 314 to 425 or 330 to 425 of SEQ ID NO:4.

Examples of the amino acid sequence of the GDH electron transfer subunit having an identity of at least 60% to SEQ ID NO:4 include the sequence of the electron transfer subunit of each of the following: Burkholderia cenocepacia J2315 strain (WP_006482958), Burkholderia thailandensis TXDOH strain (WP_009900297), Ralstonia pickettii 12D strain (WP_012761509), Ralstonia solanacearum IPO1609 strain (WP_049281214), Burkholderia phytofirmans PsJN strain (WP_012428609), Ralstonia sp. (WP_048931829), Moraxellaceae bacterium (WP_114899536), Pseudomonas knackmussii (WP_043248520), Yersinia nurmii (WP_049596851), Edaphovirga cremea (WP_114193338), Silvimonas terrae (WP_184102397) (SEQ ID NO: 12), Zymobacter palmae (WP_120185402) (SEQ ID NO: 16), Cobetia sp. (WP_158773850) (SEQ ID NO: 20), Halomonas sp. (WP_107336529) and Ewingella americana (WP_034788317) (SEQ ID NO: 8).

The truncated type of the electron transfer subunit may have a sequence identity of not less than 60%, not less than 80%, not less than 90%, not less than 95%, or not less than 98% to the amino acid sequence of amino acids 314 to 425 or 330 to 425 of SEQ ID NO:4.

In SEQ ID NO: 8, there are the first heme-binding domain (amino acid positions 41 to 45), second heme-binding domain (amino acid positions 187 to 191), and third heme-binding domain (amino acid positions 326 to 330). Among these, the first heme-binding domain and second heme-binding domain may be deleted, or the region including the first and second heme-binding domains (amino acid positions 41 to 191) may be deleted. Examples of a truncated electron transfer subunit in which the region including the first and second heme-binding domains is deleted include a truncated electron transfer subunit composed of amino acid positions 306 to 420 or 322 to 420 of SEQ ID NO: 8.

In SEQ ID NO: 12, there are the first heme-binding domain (amino acid positions 43 to 47), second heme-binding domain (amino acid positions 191 to 195), and third heme-binding domain (amino acid positions 332 to 336). Among these, the first heme-binding domain and second heme-binding domain may be deleted, or the region including the first and second heme-binding domains (amino acid positions 43 to 195) may be deleted. Examples of a truncated electron transfer subunit in which the region including the first and second heme-binding domains is deleted include a truncated electron transfer subunit composed of amino acid positions 332 to 481 of SEQ ID NO: 12.

In SEQ ID NO: 16, there are the first heme-binding domain (amino acid positions 49 to 53), second heme-binding domain (amino acid positions 196 to 200), and third heme-binding domain (amino acid positions 340 to 344). Among these, the first heme-binding domain and second heme-binding domain may be deleted, or the region including the first and second heme-binding domains (amino acid positions 49 to 200) may be deleted. Examples of a truncated electron transfer subunit in which the region including the first and second heme-binding domains is deleted include a truncated electron transfer subunit composed of amino acid positions 340 to 433 of SEQ ID NO: 16.

In SEQ ID NO: 20, there are the first heme-binding domain (amino acid positions 84 to 88), second heme-binding domain (amino acid positions 231 to 235), and third heme-binding domain (amino acid positions 418 to 422). Among these, the first heme-binding domain and second heme-binding domain may be deleted, or the region including the first and second heme-binding domains (amino acid positions 84 to 235) may be deleted. Examples of a truncated electron transfer subunit in which the region including the first and second heme-binding domains is deleted include a truncated electron transfer subunit composed of amino acid positions 418 to 522 of SEQ ID NO: 20.

The amino acid sequence of the electron transfer subunit may contain not only the mutation(s) that introduces cysteine for disulfide bond formation with a subunit and/or γ subunit (for example, D383 and Y349C, US2022/0306996A1) but also additional mutations.

### <Hitchhiker Subunit>

The glucose dehydrogenase also includes a hitchhiker subunit. The hitchhiker subunit is also called Y subunit, and has a function of forming a complex with the a subunit and being secreted into periplasm.

The hitchhiker subunit is not limited as long as it has the function. γ subunits derived from various organisms, including known γ subunits, may be used. The hitchhiker subunit may be derived from the same microorganism as the microorganism from which the catalytic subunit or electron transfer subunit is derived. Examples of the hitchhiker subunit include the hitchhiker subunit of GDH of Burkholderia cepacia. SEQ ID NO:2 is an amino acid sequence of the GDH hitchhiker subunit of the Burkholderia cepacia KS1 strain. The following description is given with reference to this sequence.

Polypeptides having some degree of sequence identity to SEQ ID NO: 2 can be used for preparing a hitchhiker subunit of CNTBP-GDH as long as they maintain the function of the hitchhiker subunit. The function of the hitchhiker subunit means a function of forming a complex with the catalytic subunit and the electron transfer subunit and being secreted into periplasm.

For example, hitchhiker subunit having sequence identity of not less than 60%, not less than 80%, not less than 90%, not less than 95%, or not less than 98% to the amino acid sequence of SEQ ID NO:2 can be used to prepare a hitchhiker subunit of the modified glucose dehydrogenase.

Examples of the amino acid sequence of the GDH hitchhiker subunit having an identity of at least 60% to SEQ ID NO:2 include the sequence of the γ subunit of each of the following: Burkholderia cenocepacia J2315 strain (WP_006482974), Burkholderia thailandensis TXDOH strain (WP_009900299), Ralstonia pickettii 12D strain (WP_012761507), Ralstonia solanacearum IPO1609 strain (WP_003265142), Burkholderia phytofirmans PsJN strain (WP_012428611), Ralstonia sp. (WP_048931990), Moraxellaceae bacterium (WP_114899538), Pseudomonas knackmussii (WP_043248523), Yersinia nurmii (WP_049596849), Edaphovirga cremea (WP_114193340), Silvimonas terrae (WP_184102399) (SEQ ID NO: 12), Zymobacter palmae (WP_051523873) (SEQ ID NO: 16), Cobetia sp. (WP_158773852) (SEQ ID NO: 20), Halomonas sp. (WP_107334715) and Ewingella americana (WP_034788279) (SEQ ID NO: 8).

The amino acid sequence of the hitchhiker subunit may contain not only the mutation that introduces cysteine for disulfide bond formation with β subunit (for example, K155C, US2022/0306996A1) but also additional mutations.

In one embodiment, CNTBP-GDH may be obtained by adding (fusing) a CNTBP to at least one subunit of these GDHs.

An example of CNTBP includes the peptide comprising the amino acid sequence of SEQ ID NO: 21 shown below. As long as retaining CNT binding ability, one or two amino acid residue may be substituted, deleted, and/or inserted in the amino acid sequence of SEQ ID NO: 21. Here, amino acid substitution is preferably a conserved substitution between similar kinds of amino acids.

(SEQ ID NO: 21)
NH₂-HMGLTKIHYSAL-COOH

CNTBP may be added to an N-terminal portion or a C-terminal portion of each subunit. One or more (two or three) CNTBP may be added to each subunit. CNTBP may be added to an N-terminus or a C-terminus. Alternatively, CNTBP may be inserted to an N-terminal portion or a C-terminal portion of each subunit. CNTBP may be directly added or added via a peptide linker. In such a case, the length of the peptide linker may be one to 20 amino acids, One to 10 amino acids, or on to five amino acids.

CNTBP may be added to at least one (for example, one or two or three) of the catalytic subunit, the electron transfer subunit, and the hitchhiker subunit. More repeated sequences or homologue sequences resulted higher affinity toward CNTBP. CNTBP may be added to one of the catalytic subunit, the electron transfer subunit, and the hitchhiker subunit, and preferably added to an electron transfer subunit and more preferably added to an N-terminal portion or a C-terminal portion of the electron transfer subunit.

In one embodiment, CNTBP-GDH has a catalytic subunit having an amino acid sequence at least 60%, at least 70%, at least 80%, at least 90%, or at least 95% identical to SEQ ID NO: 3, an electron transfer subunit having an amino acid sequence at least 60%, at least 70%, at least 80%, at least 90%, or at least 95% identical to full length or amino acids 330-425 of SEQ ID NO: 4, and a hitchhiker subunit having an amino acid sequence at least 60%, at least 70%, at least 80%, at least 90%, or at least 95% identical to SEQ ID NO: 2 and CNTBP is bound to the catalytic subunit, preferably via its N-terminus or C-terminus.

In one embodiment, CNTBP-GDH has a catalytic subunit having an amino acid sequence at least 60%, at least 70%, at least 80%, at least 90%, or at least 95% identical to SEQ ID NO: 3, an electron transfer subunit having an amino acid sequence at least 60%, at least 70%, at least 80%, at least 90%, or at least 95% identical to full length or amino acids 330-425 of SEQ ID NO: 4, and a hitchhiker subunit having an amino acid sequence at least 60%, at least 70%, at least 80%, at least 90%, or at least 95% identical to SEQ ID NO: 2 and CNTBP is bound to the electron transfer subunit, preferably via its N-terminus or C-terminus.

In one embodiment, CNTBP-GDH has a catalytic subunit having an amino acid sequence at least 60%, at least 70%, at least 80%, at least 90%, or at least 95% identical to SEQ ID NO: 3, an electron transfer subunit having an amino acid sequence at least 60%, at least 70%, at least 80%, at least 90%, or at least 95% identical to full length or amino acids 330-425 of SEQ ID NO: 4, and a hitchhiker subunit having an amino acid sequence at least 60%, at least 70%, at least 80%, at least 90%, or at least 95% identical to SEQ ID NO: 2 and CNTBP is bound to the hitchhiker subunit, preferably via its N-terminus or C-terminus.

The modified GDH (CNTBP-GDH) according to one embodiment of the present invention can be obtained by genetic engineering. More specifically, the modified GDH can be obtained by adding a CNTBP coding sequence (SEQ ID NO: 22) to at least one gene encoding each subunit of GDH to obtain a gene(s) encoding three subunits of CNTBP-GDH, and expressing the resulting CNTBP-GDH gene(s) in an arbitrary host cell or cell-free translation system. CNTBP-GDH is preferably isolated and/or purified.

The gene encoding the GDH catalytic (a) subunit is not limited as long as it has a nucleotide sequence corresponding to the amino acid sequence of the GDH a subunit. Specific examples of the gene include a DNA composed of nucleotide positions 764 to 2380 of SEQ ID NO: 1. The α-subunit gene may be a DNA having the nucleotide sequence composed of nucleotide positions 764 to 2380 of the nucleotide sequence of SEQ ID NO: 1, or a DNA which hybridizes, under stringent conditions, with a probe that can be prepared from this sequence, which DNA encodes a protein having the GDH activity, such as a DNA having a nucleotide sequence with at least 80%, 90%, or 95% identity to nucleotides 764 to 2380 of SEQ ID NO: 1.

Specific examples of the gene encoding the GDH electron transfer (B) subunit include a DNA containing the nucleotide sequence composed of nucleotide positions 2386 to 3660 of SEQ ID NO: 1. The β-subunit gene may be a DNA having the nucleotide sequence composed of nucleotide positions 2386 to 3660 of SEQ ID NO: 1, or a DNA which hybridizes, under stringent conditions, with a probe that can be prepared from this sequence, which DNA encodes a protein capable of functioning as the β subunit, such as a DNA having a nucleotide sequence with at least 80%, 90%, or 95% identity to nucleotides 2386 to 3660 of SEQ ID NO:1.

Specific examples of the gene encoding the GDH hitchhiker subunit (γ) subunit include a DNA containing the nucleotide sequence composed of nucleotide positions 258 to 761 of SEQ ID NO:1. The γ-subunit gene may be a DNA having the nucleotide sequence composed of nucleotide positions 258 to 761 of SEQ ID NO: 1, or a DNA which hybridizes, under stringent conditions, with a probe that can be prepared from this sequence, which DNA encodes a protein capable of functioning as the γ subunit, such as a DNA having a nucleotide sequence with at least 80%, 90%, or 95% identity to nucleotides 258 to 761 of SEQ ID NO: 1.

Examples of the stringent conditions described above include conditions that allow hybridization of DNAs having an identity of preferably at least 80%, more preferably at least 90%, especially preferably at least 95%, with each other and does not allow hybridization of DNAs having an identity of less than 80%, less than 90% or less than 95%. Specific examples of the stringent conditions include those in which washing is carried out with 0.1×SSC and 0.1% SDS at 60° C., 65° C., or 68° C.

The α-subunit gene, β-subunit gene, and γ-subunit gene can be obtained by, for example, PCR using chromosomal DNA of the Burkholderia cepacia KS1 strain as a template. The PCR primers therefor can be prepared by chemical synthesis based on the above-described nucleotide sequences. Alternatively, the genes can be obtained from chromosomal DNA of the Burkholderia cepacia KS1 strain by hybridization using, as probes, oligonucleotides prepared based on the above-described sequences. Strains other than the KS1 strain, such as the Burkholderia cenocepacia J2315 strain, Burkholderia thailandensis TXDOH strain, Ralstonia pickettii 12D strain, Ralstonia solanacearum IPO1609 strain, and Burkholderia phytofirmans PsJN strain, may also be used.

CNTBP-coding sequence (e.g., SEQ ID NO: 22) can be added to a gene encoding each subunit of GDH by a conventional genetic engineering such as PCR. The addition of the CNTBP-coding sequence to the DNA encoding each subunit may be carried out by using each gene encoding each subunit or by using a polycistronic DNA fragment encoding the γ subunit, a subunit, and β subunit in this order.

The polycistronic DNA fragment encoding the γ subunit, a subunit, and β subunit in this order can be obtained by, for example, PCR using chromosomal DNA of the Burkholderia cepacia KS1 strain as a template, and oligonucleotides having the nucleotide sequences of SEQ ID NOs: 23 and 24 as primers.

The vector used for obtaining the genes of the GDH subunits, introducing the mutations, expressing the genes, and the like include vectors capable of functioning in bacteria belonging to the genus Escherichia, such as pTrc99A, pBR322, pUC18, pUC118, pUC19, pUC119, pACYC184, and pBBR122. Examples of the promoter to be used for the gene expression include lac, trp, tac, trc, PL, tet, and PhoA. By introducing the γ subunit gene, a subunit gene, and β subunit gene to an appropriate site in an expression vector containing a promoter, insertion of the genes into the vector and linking of the promoter can be carried out in the same single process. Examples of such an expression vector include pTrc99A, pBluescript, and pKK223-3.

The γ subunit gene, a subunit gene, and β subunit gene may be incorporated in the chromosomal DNA of the host microorganism in a form which allows the expression. Examples of the method of transformation of the microorganism with the recombinant vector include the competent cell method by calcium treatment, the protoplast method, and the electroporation method.

Examples of the host microorganism include, but are not limited to, bacteria belonging to the genus Bacillus such as Bacillus subtilis; yeasts such as Saccharomyces cerevisiae; and filamentous fungi such as Aspergillus niger, A. oryzae. Any host microorganism suitable for production of a foreign protein may be used.

The transformant such as a microorganism transformed with the CNTBP-GDH genes may be cultured by an appropriate culture method depending on the type of the transformant. Examples of the conditions for the culture include culturing at 30 to 37° C. for 12 to 24 hours.

The CNTBP-GDH may be used also after purification. The CNTBP-GDH may contain an additional sequence such as a tag sequence for purification.

CNTBP-GDH may be prepared by using GDH from other microorganisms than B. cepacian in the similar manner.

In one embodiment, the above-mentioned CNTBP-GDH is used as a component of a glucose sensor. The glucose sensor according to one embodiment of the invention comprises an electrode which comprises a carbon nanotube (CNT) and the CNTBP-GDH wherein the CNTBP-GDH is bound to the carbon nanotube.

In a preferable embodiment, CNT is coated or immobilized on the surface of the electrode and CNTBP-GDH is bound to the CNT coated or immobilized on the electrode surface. By using CNTBP-GDH, the orientation of the GDH on the electrode is controlled which leads to an improvement of the enzyme electrode such as a conciseness of the measurement ability.

Such an enzyme electrode comprising CNT and CNTBP-GDH may be prepared by coating CNT on a surface of an electrode material and then adding a solution containing CNTBP-GDH thereon followed by drying. CNT may be coated on the surface of the electrode by a conventional technique such as screen printing.

The electrode material may be a metallic material such as gold (Au), platinum (Pt), silver (Ag), and palladium (Pd) or a carbon material. The electrode may be provided on an insulating substrate formed with an insulating material, wherein examples of the insulating material include resins (plastics) such as thermoplastic resins including polyetherimide (PEI), polyethylene terephthalate (PET), and polyethylene (PE), as well as polyimide resins and epoxy resins; glasses; ceramics; and papers.

The CNTBP-GDH according to one embodiment of the present invention may be used as a component of an enzyme electrode of a glucose sensor. Specific examples of the glucose sensor include glucose sensors that use, as a working electrode, an enzyme electrode formed by immobilization of the CNTBP-GDH on a CNT-modified surface of an electrode such as a gold electrode, platinum electrode, or carbon electrode. The sensor means a measurement system for electrochemically measuring the concentration of a test substance of interest, and usually contains three electrodes, which are a working electrode (enzyme electrode), counter electrode (platinum or the like), and reference electrode (Ag/AgCl or the like). The sensor may also be a two-electrode system constituted by a working electrode and a counter electrode, such as those used in conventional, simple blood glucose level systems. The sensor preferably further contains: a constant-temperature cell in which a buffer and a test sample are to be placed; a power source for applying a voltage to the working electrode; an ammeter; a recorder; and/or the like. The sensor may be either a batch-type sensor or flow-type sensor. In particular, the flow-type sensor may be a sensor capable of continuous measurement of the blood glucose level. More specifically, the sensor may be a sensor having a two-electrode system or three-electrode system on which the enzyme of the present invention is immobilized, which electrode system is inserted into a blood sample or dialysis sample that is continuously supplied, or into blood or interstitial fluid, to perform the measurement. The structure of such an enzyme sensor is well known in the art, and described in, for example, Biosensors-Fundamental and Applications-Anthony P. F. Turner, Isao Karube and George S. Wilson, Oxford University Press 1987.

The sensor according to one mode of the present invention may be a direct electron transfer-type sensor containing no electron-transfer mediator.

The measurement of the glucose concentration using the glucose sensor according to the invention may be carried out as follows. A buffer is placed in a constant-temperature cell of the sensor, and the temperature of the cell is kept constant. As a working electrode, an enzyme electrode comprising the CNTBP-GDH and CNT is used. As a counter electrode, for example, a platinum electrode is used. As a reference electrode, for example, an Ag/AgCl electrode is used. A constant voltage is applied to the working electrode. The voltage may be −100 mV or more, +10 mV or more, or +100 mV or more, with respect to the Ag/AgCl electrode. The upper limit of the voltage may be, but not limited to, +5000 mV, +2000 mV, +1500 mV or +1000 mV with respect to the Ag/AgCl electrode. After the electric current becomes constant, a sample containing glucose is placed in the constant-temperature cell, and the increase in the electric current is measured. According to a calibration curve prepared using glucose solutions having standard concentrations, the glucose concentration in the sample can be calculated.

### EXAMPLES

### Materials and Methods

### Construction of Expression Vectors

FADGDH from B. cepacia was used. The α-subunit contains QYY mutations as well as P205C mutation for disulfide formation with β-subunit (US2022/0306996A1). The β-subunit is a truncated type (amino acids 314 to 425 of SEQ ID NO: 4) and contains D383C and Y349C mutations for disulfide formation with α-subunit and γ-subunit (US2022/0306996A1). The γ-subunit contains K155C mutation for disulfide formation with β-subunit (US2022/0306996A1).

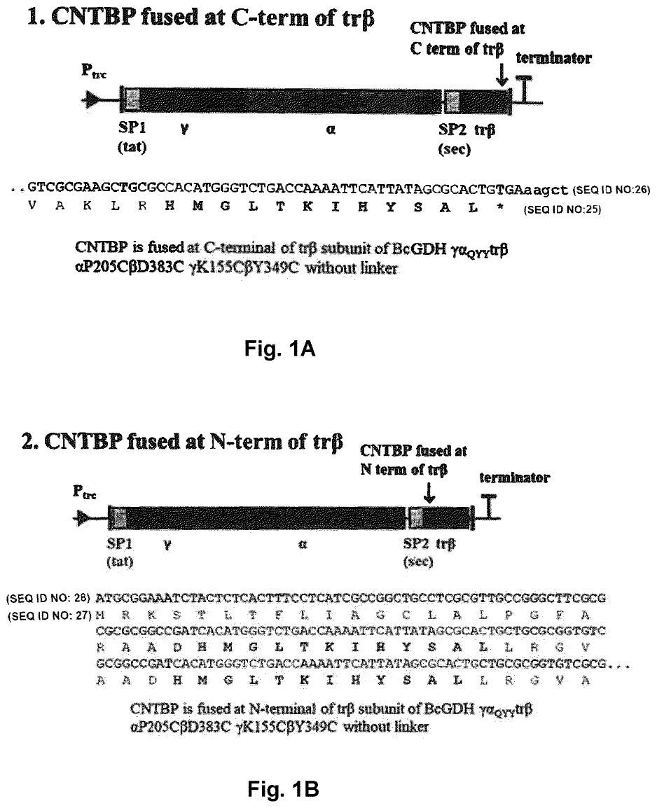

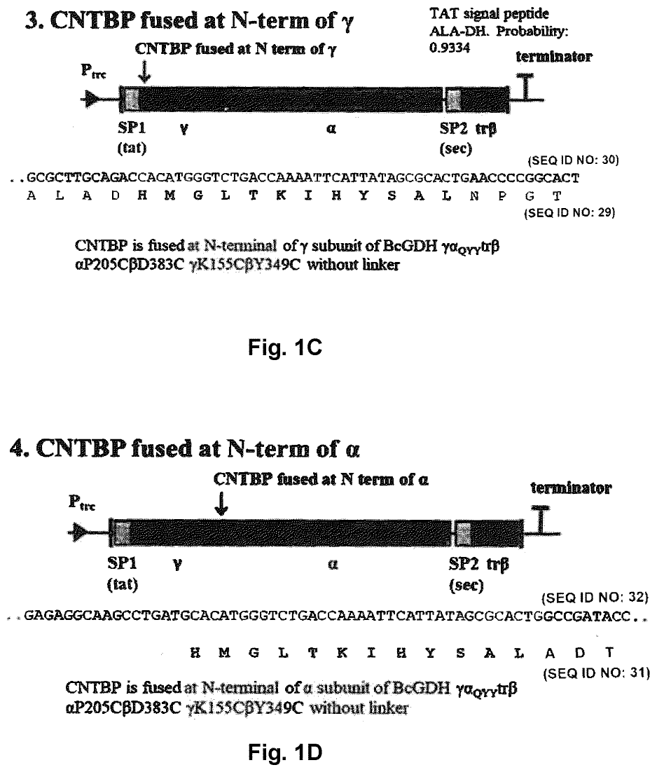

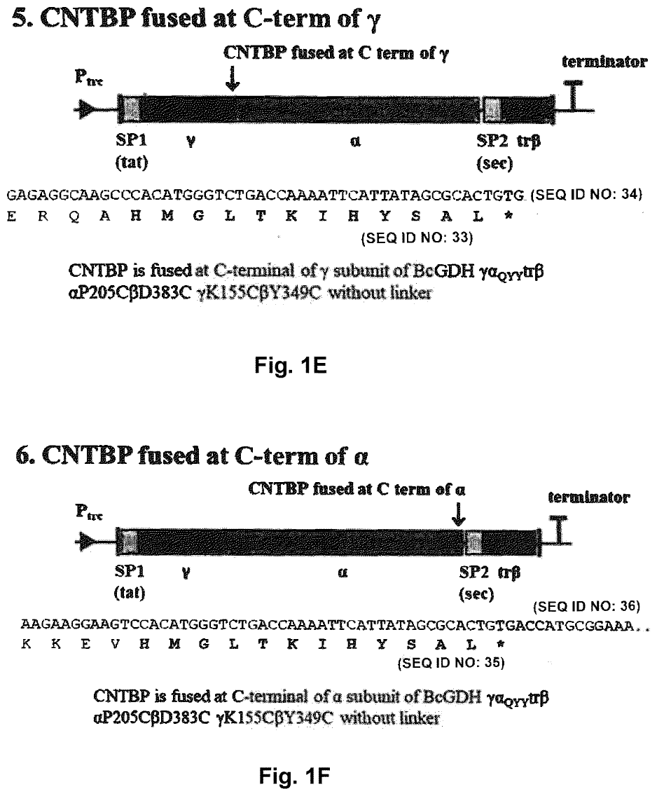

Carbon nanotube binding peptide (CNTBP) was added to the N and C-terminal of each of the subunits as shown in FIGS. 1A to IF.

The resulting constructed modified FADGDH genes include:

### 1) FADGDH-γαtrβ_{C}-CNTBP

CNTBP was fused at C-terminus of the truncated β-subunit.

### 2) FADGDH-γαtrβ_{N}-CNTBP

Two CNTBPs were fused at N-terminus of the truncated β-subunit. The N-term portion (amino acids 1 to 27 of SEQ ID NO: 4) was included for secretion and post translational modification but the N-term portion is cleaved upon maturation.

### 3) FADGDH-γ_{N}-CNTBP-αtrβ

CNTBP was inserted to N-terminal portion (between D48 and N49) of the γ-subunit.

### 4) FADGDH-γα_{N}-CNTBP-trβ

CNTBP was fused at N-terminus of the α-subunit.

### 5) FADGDH-γ_{C}-CNTBP-αtrβ

CNTBP was fused at C-terminus of the γ-subunit.

### 6) FADGDH-γα_{C}-CNTBP-trβ

CNTBP was fused at C-terminus of the α-subunit.

### Preparation of Genes and Recombinant Production

Constructed the modified FADGDH genes containing CNTBP sequence were inserted into the expression vector pTrc99A. The resulting plasmids were introduced into Escherichia coli BL21 (DE3) harboring the pBBJMccm vector in order to express the E. coli ccmABCDEFGH genes essential for the maturation of cytochrome c. The resulting transformants were grown on an LB agar plate containing 100 μg/mL ampicillin and 50 μg/mL kanamycin.

The transformants were precultured in 3 mL of LB medium with 100 μg/mL ampicillin and 50 μg/mL kanamycin for 17 h at 37° C. and a rotation rate of 250 rpm. 1% inoculation was performed utilizing 1 mL of preculture to 100 mL of ZYP-5052 medium (LB medium (Peptone140) with 0.5% glycerol, 0.05% glucose, 0.2% lactose, 50 mM KH₂PO₄, 25 mM (NH₄) PO₄, 50 mM Na₂HPO₄, and MgSO₄*) containing 100 μg/mL of ampicillin and 50 μg/mL kanamycin, and were cultivated for 30 h at 30° C. and a rotation rate of 130 rpm. Cells were harvested and washed with saline (0.85% NaCl). Harvested cells were stored at −80° C. until disruption.

For preparation of the crude extract, cultures were harvested and disrupted by suspending and centrifuging harvested cells in 1× BugBuster® Protein Extraction Reagent (Millipore sigma 70921). The resulting soluble fractions were subjected to DC Protein Assay for protein quantification.

### Protein Purification

The cells were harvested by centrifugation and resuspended in lysis buffer. The cells were then disrupted by sonication, and the cell debris was removed by centrifugation. The supernatant, containing the soluble modified FADGDH, was first subjected to anion exchange chromatography to separate the protein based on its charge. The fractions containing the modified FADGDH were pooled and then subjected to size exclusion chromatography to further purify the protein based on its size. The purified protein was then concentrated for further analysis.

### Anion Exchange Chromatography (AEC)

Column: HiTrapQ
A buffer: 10 mM HEPES pH 7.8
B buffer: 0.5 M NaCl, 10 mM HEPES pH 7.8
B 0-40%/30 CV, 100% 5 CV
   Flow rate: 1 mL/min

### Size Exclusion Chromatography (SEC)

Column: Superdex Increase 10/300
Elution buffer: PBS
Flow rate: 0.5 mL/min
Fractionation: 0.5 mL

### Confirmation of Modified FAGDH Production

The production of the modified FADGDH was confirmed by SDS-PAGE. The enzyme activity of the modified FADGDH was also measured and compared with that of the wild-type enzyme. Enzyme activity was assayed with the PMS/DCIP and Ru/MTT method to evaluate dehydrogenase enzyme activity. For the PMS/DCIP method, the enzyme sample was incubated in a 20 mM potassium phosphate buffer (pH 7.0) containing 0.6 mM phenazine methosulfate (CAS 299-11-6), 0.06 mM 2,6-dichlorophenol-indophenol sodium salt dihydrate (CAS 620-45-1), 0.1% Triton, and 100 mM glucose. The activity was determined by monitoring the increase in the absorbance at 600 nm and using the molar absorption coefficient of formazan (16.3 mM cm-1 at pH 7.0). For the Ru/MTT method, the enzyme sample was incubated in a 20 mM potassium phosphate buffer (pH 7.0) containing 2% (w/v) hexaammineruthenium (III) chloride (CAS 14282-91-8), 1 mM MTT (CAS 298-93-1), 0.2% Triton and 100 mM glucose. The activity was determined by monitoring the increase in the absorbance at 565 nm and using the molar absorption coefficient of formazan (20 mM cm-1 at pH 7.0). One unit of enzyme activity is defined as the amount of enzyme that oxidizes 1 μmol of glucose per min.

### Preparation of Working Electrode:

Two different types of commercially available electrodes were prepared as the working electrode: multi-walled carbon nanotube (MWCNT)-modified screen-printed carbon electrodes (DropSens 110CNT), and bare screen-printed carbon electrodes (DropSens C110). 3 μL of 85 ng/μL FADGDH (FADGDH-γαtrβ_{C}-CNTBP, FADGDH-γαtrβ_{N}-CNTBP, and FADGDH-γαtrβ as a control) was prepared in 10 mM HEPES buffer pH 7.8 and dropped onto each respective working electrode and incubated at 4° C. overnight. The working electrodes were rinsed with 100 mM P.P.B. for electrochemical measurement.

### Electrochemical Characterization

Using cyclic voltammetry, the constructed working electrodes were evaluated with Ag/AgCl and Carbon (SPCE) as reference and counter electrodes, respectively. An applied potential is cycled between −0.5 to +0.5 V at a sweep rate of 20 mV/sec. Glucose concentration was 0, 1, 5, and 20 mM. The current responses were recorded and analyzed for highest current response.

### Results

### Construction and Production of the Modified FADGDH:

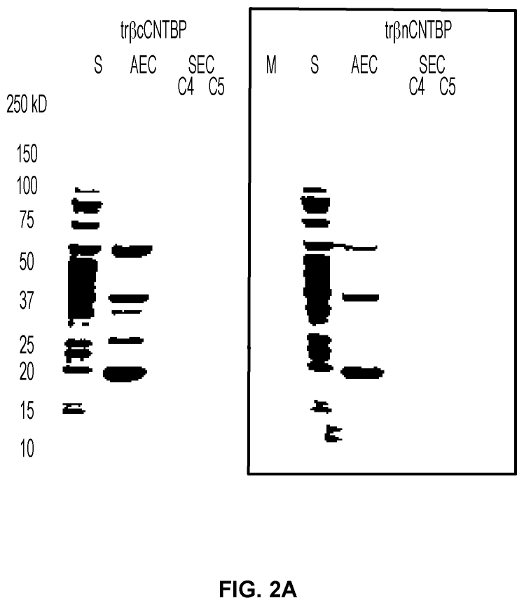

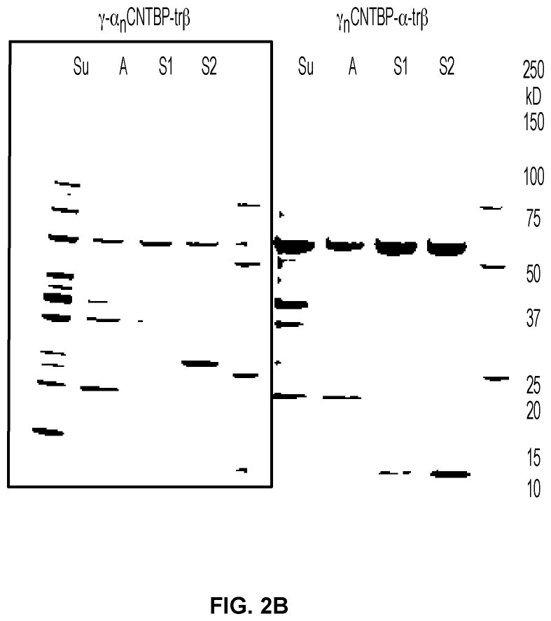

The CNTBP fusion was successfully introduced into the FADGDH gene using site-directed mutagenesis. The modified FADGDH gene was cloned into a plasmid, which was then transformed into E. coli cells for protein expression. The modified FADGDHs (FADGDH-γαtrβ_{C}-CNTBP, FADGDH-γαtrβ_{N}-CNTBP, FADGDH-γ_{N}-CNTBP-αtrβ, FADGDH-γα_{N}-CNTBP-trβ) were successfully purified from the bacterial cells using a combination of anion exchange chromatography and size exclusion chromatography. The purity of each enzyme modified FADGDHs was confirmed by SDS-PAGE (FIGS. 2A and 2B), which showed a bands corresponding to each expected subunit (γ, α, and trβ). The modified FADGDHs with CNTBP fused to the C-terminal of γ- or α-subunits were not further investigated due to low crude enzyme activity and purification yield.

The enzyme activity of the modified FADGDHs was found to be comparable to that of the wild-type enzyme, indicating that the mutation did not adversely affect the enzyme's function.

TABLE 1
Enzyme activity of each CNTBP-FADGDH

| | Ru/MTT U/mg | Ru/MTT U/mg |
|---|---|---|
| BcGDH | (crude) | (purified) |
| γαtrβ (control) | 3.86 | 103.6 |
| γαtrβ_{C}-CNTBP | 2.78 | 180 |
| γαtrβ_{N}-CNTBP | 1.90 | 155 |
| γ_{N}-CNTBP-αtrβ | 5.9 | 238 |
| γα_{N}-CNTBP-trβ | 4.8 | 98 |
| γ_{C}-CNTBP-αtrβ | 0.52 | 3.47 |
| γα_{C}-CNTBP-trβ | 0.46 | — |

Glucose conc. 100 mM, 2% Ru/1 mM MTT

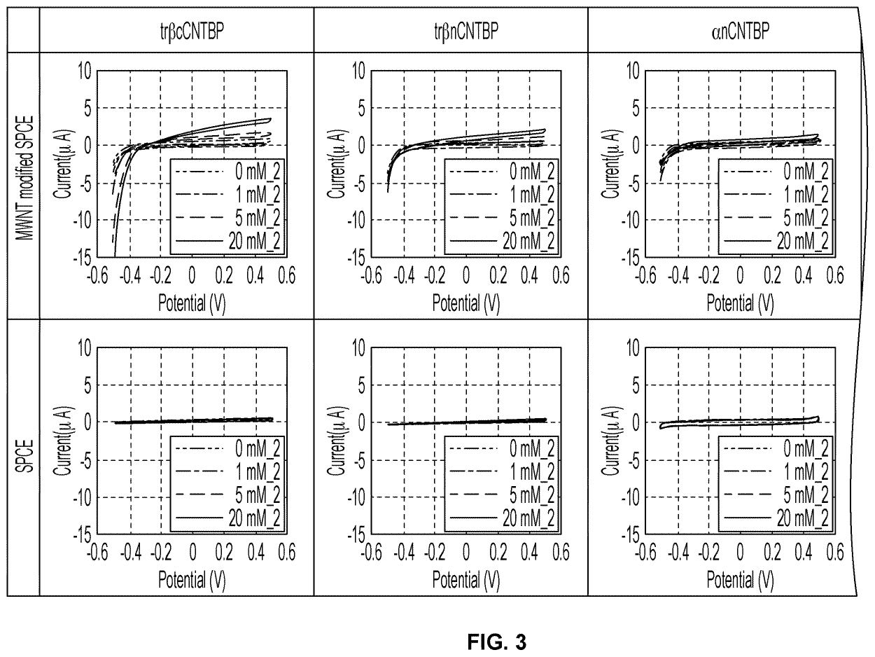

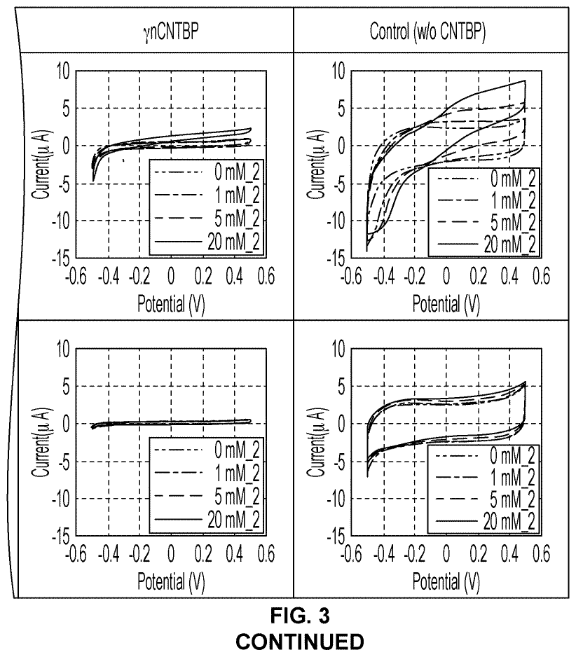

Fusing CNTBP on the FADGDH was found to effectively control the orientation of the enzyme on the electrode surface. This was confirmed by comparing the cyclic voltammetry results of the modified FADGDHs with those of randomly oriented BcGDH containing no CNTBP (FIG. 3).
Oxidative current: trβcCNTBP>trβnCNTBP>γnCNTBP>αnCNTBP

The modified FADGDHs showed much less hysteresis, indicating better enzyme orientation.

The highest oxidative current in the presence of glucose indicates that the highest response current in CA is expected by trβcCNTBP.

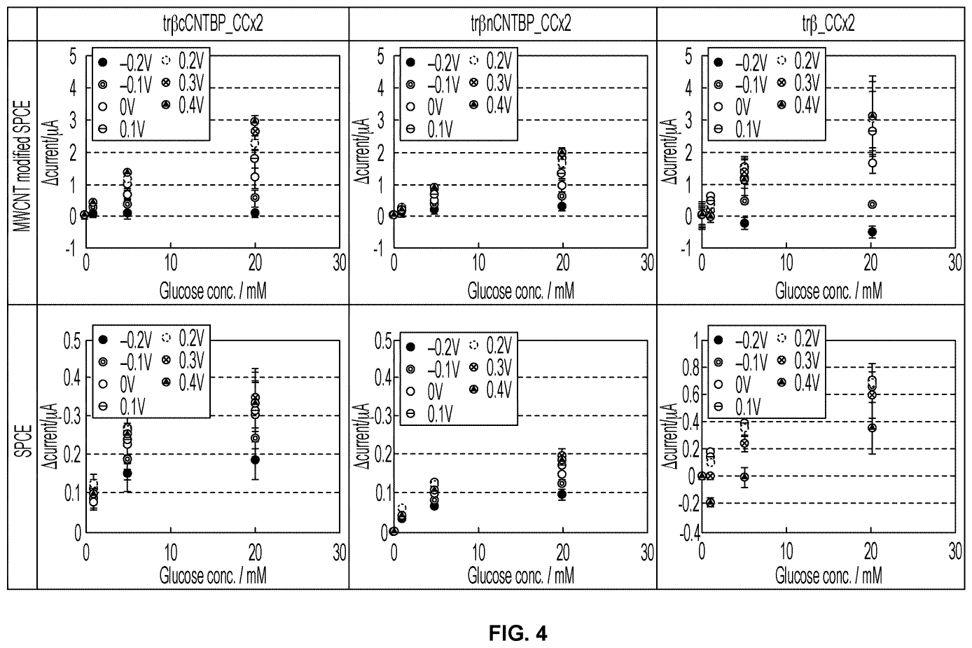

FADGDH-γαtrβ_{C}-CNTBP, and FADGDH-γαtrβN-CNTBP showed glucose concentration dependence of current values at each potential (FIG. 4).

Fusing CNTBP at the C terminal at the trβ-subunit of FADGDH with controlled orientation on the electrode surface exhibits improved DET ability, making it a promising candidate for use in bio-electrochemical applications such as glucose monitoring. This mutant not only maintained similar enzymatic activity to the control but also demonstrated enhanced direct electron transfer (DET) ability in the presence of MWCNT electrodes, compared to the other mutants, suggesting that the fusion plays a crucial role in achieving efficient electron transfer.

Comparing the performance of the enzyme with controlled orientation to that with random orientation, it is evident that controlling the orientation of the enzyme offers significant benefits. The controlled orientation ensures that the redox center of the enzyme is optimally positioned for DET, leading to improved efficiency of electron transfer. This, in turn, translates into higher current densities, which can lead to improved accuracy and sensitivity in bio-electrochemical applications such as glucose monitoring.

Our study provides compelling evidence that the CNTBP fusion on the modified FADGDH does indeed allow for control over the enzyme's orientation on the electrode surface. The cyclic voltammetry results clearly demonstrated that the modified enzyme, with its fusion to CNTBP, exhibited much less hysteresis compared to randomly oriented BcGDH. This indicates a more efficient electron transfer, which is consistent with an optimal enzyme orientation.

In conclusion, our findings underscore the potential of engineering enzymes like FADGDH to control their orientation on electrode surfaces, thereby maximizing their DET ability. This opens up new avenues for the design of more efficient bio-electrochemical devices.

The contents of all publications including patents and patent applications as well as non-patent documents cited in the present description are hereby incorporated by reference to the same extent as if all contents were clearly described.

1. A modified glucose dehydrogenase comprising a catalytic subunit, an electron transfer subunit, and a hitchhiker subunit, wherein a carbon nanotube binding peptide is fused to at least one of the catalytic subunit, the electron transfer subunit, and the hitchhiker subunit.

2. The modified glucose dehydrogenase according to claim 1, wherein said carbon nanotube binding peptide comprises the amino acid sequence of SEQ ID NO:21.

3. The modified glucose dehydrogenase according to claim 1, wherein said electron transfer subunit is a truncated electron transfer subunit.

4. The modified glucose dehydrogenase according to claim 1, wherein said carbon nanotube binding peptide is fused to said electron transfer subunit.

5. The modified glucose dehydrogenase according to claim 1, wherein said carbon nanotube binding peptide is fused to N-terminal portion or C-terminal portion of at least one of the catalytic subunit, the electron transfer subunit, and the hitchhiker subunit.

6. The modified glucose dehydrogenase according to claim 1, wherein said modified glucose dehydrogenase is from Burkholderia cepacia.

7. An enzyme electrode comprising the modified glucose dehydrogenase according to claim 1 and carbon nanotube.

8. A glucose sensor comprising the enzyme electrode according to claim 7.

9. A method of measuring glucose comprising measuring glucose by using the glucose sensor according to claim 8.

10. The method according to claim 9, wherein voltage of at least −0.2V with respect to Ag/AgCl electrode is applied to the enzyme electrode.
